(19) United States
(12) Patent Application Publication
(10) Pub. No.: US 2009/0170953 A1
(43) Pub. Date: Jul. 2, 2009

(54) INDENTIFICATION OF COMPOUNDS THAT INHIBIT REPLICATION OF HUMAN IMMUNODEFICIENCY VIRUS

(75) Inventors: Jan Maria Balzarini, Heverlee (BE); Anders Vahlne, Stockholm (SE); Marita Hogberg, Tullinge (SE); Weimin Tong, Uppsala (SE)

Correspondence Address: KNOBBE MARTENS OLSON & BEAR LLP, 2040 MAIN STREET, FOURTEENTH FLOOR, IRVINE, CA, 92614, US

(73) Assignee: Tripep AB, Huddinge (SE)

(21) Appl. No.: 11/929,695

(22) Filed: Oct. 30, 2007

Related U.S. Application Data
(62) Division of application No. 10/783,053, filed on Feb. 19, 2004, now abandoned.
(60) Provisional application No. 60/505,217, filed on Sep. 22, 2003.
(60) Provisional application No. 60/493,893, filed on Aug. 8, 2003.
(60) Provisional application No. 60/449,494, filed on Feb. 21, 2003.

(51) Int. Cl. A61K 31/164; C12N 7/04; A61P 31/18

(52) U.S. Cl. 514/626; 435/235.1

(57) ABSTRACT

The present invention relates to the discovery of a novel class of compounds that inhibit the replication of human immunodeficiency virus (HIV) and approaches to identify these compounds. More specifically, it has been found that enzymatically prepared alpha-hydroxyglycinamide and synthetically prepared alpha-hydroxyglycinamide inhibit the replication of HIV in human serum. Embodiments include methods to identify modified glycinamide compounds that inhibit HIV, methods to isolate and synthesize modified glycinamide compounds, and therapeutic compositions comprising these compounds.

### CROSS-REFERENCE OF RELATED APPLICATIONS

This application is a divisional of U.S. application Ser. No. 10/783,053, filed Feb. 19, 2004, which claims priority to U.S. Provisional Application No. 60/449,494, filed Feb. 21, 2003, U.S. Provisional Application No. 60/493,893, filed Aug. 8, 2003, and U.S. Provisional Application No. 60/505,217, filed Sep. 22, 2003, the disclosures of which are all hereby expressly incorporated by reference in their entireties.

### FIELD OF THE INVENTION

A new class of drags that inhibit the replication of human immunodeficiency virus (HIV) has been discovered. Several methods to identify metabolites of glycinamide that inhibit the replication of HIV are described. Embodiments include methods to identify and synthesize modified glycinamide compounds and compositions comprising modified glycinamide compounds.

### BACKGROUND OF THE INVENTION

Human immunodeficiency virus (HIV) is the name given to a lentivirus that infects humans and that causes acquired immuno-deficiency syndrome (AIDS). HIV is a complex retrovirus containing at least nine genes. The viral structural genes, designated gag, pol, and env, respectively code for inter alia the viral core proteins, reverse transcriptase, and the viral glycoproteins of the viral envelope. The remaining HIV genes are accessory genes involved in viral replication. The gag and env genes encode polyproteins, i.e., the proteins synthesized from each of these genes are post-translationally cleaved into several smaller proteins.

Although the overall shape of HIV is spherical, the nucleocapsid is asymmetrical having a long dimension of about 100 nm, a wide free end about 40-60 nm, and a narrow end about 20 nm in width. The nucleocapsid within each mature virion is composed of two molecules of the viral single-stranded RNA genome encapsulated by proteins proteolytically processed from the Gag precursor polypeptide. Cleavage of the gag gene polyprotein Pr55^{gag} by a viral coded protease (PR) produces mature capsid proteins.

Since the discovery of HIV-1 as the etiologic agent of AIDS, significant progress has been made in understanding the mechanisms by which the virus causes disease. While many diagnostic tests have been developed, progress in HIV vaccine therapy has been slow largely due to the heterogeneous nature of the virus and the lack of suitable animal models. (See e.g., Martin, Nature, 345:572-573 (1990)).

A variety of pharmaceutical agents have been used in attempts to treat AIDS. HIV reverse transcriptase (RT) is one drug target because of its crucial role in viral replication, however, many, if not all, of the drugs that inhibit the enzyme are limited in their usefulness as therapeutic agents. These are nucleoside/nucleotide analogue RT inhibitors (NRTI:s) that will induce chain termination and agents that directly inhibit the enzyme, referred to as non-nucleoside analogue RT inhibitors (NNRTI:s). Nucleoside derivatives, such as azidothymidine (AZT, Zidovudine®) and the other RT inhibitors cause serious side effects such that many patients cannot tolerate administration.

Another drug target is the HIV protease (PR) crucial to virus maturation. PR is an aspartic acid protease and can be inhibited by synthetic compounds. (See e.g., Richards, FEBS Lett., 253:214-216 (1989)). Protease inhibitors strongly inhibit the replication of HIV but prolonged therapy has been associated with metabolic diseases such as lipodystrophy, hyperlipidemia, and insulin resistance.

Additionally, HIV quickly develops resistance to NRTI:s, NNRT:s and protease inhibitors. Resistant virus can also spread between patients. Studies have shown, for example, that in the US one tenth to one fifth of the individuals recently infected by HIV already have virus that has developed resistance to one or more antiviral drug, probably because they were infected by a person that at the time of transmission carried a virus that had developed resistance.

Over the last decade it has been discovered that several peptide amides inhibit the replication of HIV. (See, e.g., U.S. Pat. Nos. 5,627,035; 6,258,932; 6,455,670; and U.S. patent application Ser. Nos. 09/827,822; 09/938,806; 10/072,783; 10/217,933; and 10/235,158, all of which are herein expressly incorporated by reference in their entireties). These peptides amides appear to inhibit HIV replication in a manner that is different than reverse transcriptase inhibitors and protease inhibitors and have few, if any, side-effects. Despite these efforts, the need for more selective therapeutic agents that inhibit HIV replication is manifest.

### BRIEF SUMMARY OF THE INVENTION

It has been discovered that enzymatically prepared and synthetically prepared α-hydroxyglycinamide inhibit the replication of HIV in human serum. Accordingly, aspects of the invention include therapeutic compositions that consist, consist essentially of, or comprise modified glycinamide compounds. Modified glycinamide compounds (e.g., Metabolite X, alpha hydroxyglycinamide, or AlphaHGA) in either enantiomer (L or D) or both or either isomer (R or S) or both are provided as active ingredients of pharmaceuticals and medicaments that inhibit the replication and/or propagation of HIV. Modified glycinamide compounds, such as α-hydroxyglycinamide (alpha-hydroxy-gly-NH₂), α-peroxyglycinamide dimer (NH₂-gly-O—O-gly-NH₂), diglycinamide ether (NH₂-gly-O-gly-NH₂) and alpha-methoxyglycinamide (alpha-MeO-gly-NH₂), or pharmaceutically acceptable salts thereof are the preferred active ingredients for incorporation into a pharmaceutically acceptable formulation that can be used to inhibit the replication of HIV.

Accordingly, antiretroviral pharmaceuticals and medicaments can be prepared by providing a modified glycinamide compound (e.g., a compound provided by formulas A, B, C, D, E, F, G, H, or 1) or a pharmaceutically acceptable salt thereof in either enantiomer (L or D) or both or either isomer (R or S) or both. Preferred compounds for formulation into an antiretroviral pharmaceutical or medicament include, for example, α-hydroxyglycinamide (formula C), α-peroxyglycinamide dimer (formula E), diglycinamide ether (formula F), and alpha-methoxyglycinamide, or pharmaceutically acceptable salts thereof in either enantiomer (L or D) or both or either isomer (R or S) or both. The antiretroviral pharmaceuticals and medicaments describe herein can be provided in unit dosage form (e.g., tablets, capsules, gelcaps, liquid doses, injectable doses, transdermal or intranasal doses) and can contain, in addition to the modified glycinamide compound, a pharmaceutically acceptable carrier or exipient. Containers comprising said pharmaceuticals and medicaments (e.g., sterile vials, septum sealed vials, bottles, jars, syringes, atomizers, swabs) whether in bulk or in individual doses are also embodiments and, preferably, said formulations are prepared according to certified good manufacturing processes (GMP) (e.g., suitable for or accepted by a governmental regulatory body, such as the Federal Drug Administration (FDA)) and said containers comprise a label or other indicia that reflects approval of said formulation from said governmental regulatory body. Nutriceuticals containing said compounds with or without structure-function indicia are also embodiments, however.

Some embodiments also include a precursor or prodrug for one or more of said antiretroviral compounds (e.g., Metabolite X, α-hydroxyglycinamide (formula C), α-peroxyglycinamide dimer (formula E), diglycinamide ether (formula F), and alpha-methoxyglycinamide, in either enantiomer (L or D) or both or either isomer (R or S) or both). Such precursors or prodrugs include, for example, a glycinamide containing peptide or glycinamide itself (e.g., GPG-NH₂ or ALGPG-NH₂). These precursors or prodrugs are provided in conjunction with (e.g., coadministration in a mixture or before or after delivery of the prodrug) with a material (e.g., a cofactor(s) containing material such as fetal calf serum, bovine serum, plasma, or milk, horse serum, plasma, or milk, cat or dog serum in isolated, enriched, or raw form) capable of converting the precursor or prodrug into a modified glycinamide compound (e.g., a compound provided by formulas A, B, C, D, E, F, G, H, or I) in either enantiomer (L or D) or both or either isomer (R or S) or both, such as Metabolite X). As above, said prodrug/cofactor formulations can be prepared according to certified good manufacturing processes (GMP) (e.g., suitable for or accepted by a governmental regulatory body, such as the Federal Drug Administration (FDA)) and said containers comprise a label or other indicia that reflects approval of said formulation from said governmental regulatory body. Nutriceuticals containing said formulations with or without structure-function indicia are also embodiments.

Alpha-hydroxyglycinamide (α-hydroxyglycinamide) or a pharmaceutically acceptable salt thereof (also referred to collectively as “alphaHGA”) is a preferred active ingredient for incorporation into pharmaceuticals and/or medicaments that can be used to inhibit the replication of HIV. Pharmaceuticals and medicaments that consist of, consist essentially of, or comprise L-alphaHGA (in R or S isomer) or D-alpha HGA (in R or S isomer) or both (with either R or S or both isomers) are embodiments. These compositions (e.g., ampules, capsules, pills, tablets, intravenous solutions, transdermal, intranasal solutions, and other pharmaceutically acceptable formulations) preferably contain, provide, or deliver an amount of enzymatically prepared (Metabolite X) or synthetically prepared (alphaHGA) alpha hydroxyglycinamide that inhibits the replication and/or propagation of HIV.

Embodiments include, for example, pharmaceuticals and medicaments consisting, consisting essentially of, or comprising a modified glycinamide compound of formula (A):

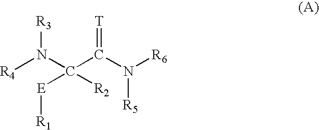

or a pharmaceutically acceptable salt, amide, ester, or prodrug thereof; wherein:

a) E is selected from the group consisting of oxygen, sulfur, and NR₇;

b) T is selected from the group consisting of oxygen, sulfur, and NR₈; and

c) R₁-R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl.

Desirable compositions include pharmaceuticals and medicaments that consist of; consist essentially of, or comprise a modified glycinamide compound of formula (B):

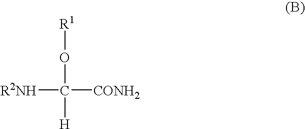

wherein, R¹ is a hydrogen atom, a lower alkyl group, a lower alkenyl group, a lower alkynyl group, a benzyl group, or a silyl group substituted with an alkyl group or an alkyl group and an aromatic group and R² is a hydrogen atom or an amino protecting group, or a salt thereof.

Preferred compositions include pharmaceuticals and medicaments that consist of, consist essentially of, or comprise a modified glycinamide compound of formula (C):

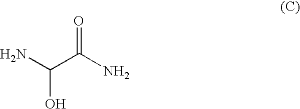

or a pharmaceutically acceptable salt, amide, ester, or prodrug thereof. Particularly preferred compositions include pharmaceuticals and medicaments that consist of, consist essentially of, or comprise a modified glycinamide salt of formula (D):

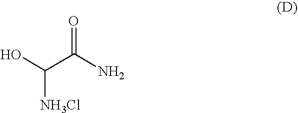

The compound of formula (C), α-hydroxyglycinamide, also referred to as Metabolite X or alphaHGA, has been produced by an enzymatic process and isolated using cation exchange HPLC and the compound of formula (D) has been made synthetically. In some contexts, both the compounds of formula (C) and (D) in either enantiomer (L or D) or both or either isomer (R or S) or both are referred to as “Metabolite X,” “alphaHGA,” or “modified glycinamide,” interchangeably.

Preferred compositions also include pharmaceuticals and medicaments that consist of, consist essentially of, or comprise a modified glycinamide compound of formula (E) or formula (F) or a pharmaceutically acceptable salt thereof:

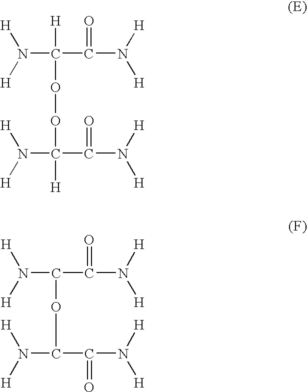

Preferred compositions also include pharmaceuticals and medicaments that consist of, consist essentially of, or comprise a modified glycinamide compound of formula (G) or a pharmaceutically acceptable salt thereof:

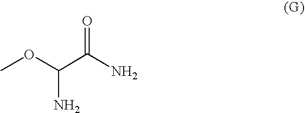

Alpha-methoxyglycinamide has also been prepared synthetically and this compound has been found to be more stable than alpha-hydroxyglycinamide.

Embodiments also include several methods to identify and isolate modified glycinamide compounds that inhibit the replication of HIV and methods to synthesize these compounds. Some embodiments concern methods to inhibit the replication and/or propagation of HIV, wherein a subject in need of an agent that inhibits the replication of HIV is provided an amount of enzymatically prepared (Metabolite X) or synthetically prepared alpha hydroxyglycinamide (alphaHGA) sufficient to inhibit the propagation or replication of the virus. In some of these methods, the affect on HIV replication is measured (e.g., by observing or monitoring a reduction in viral lode or a marker thereof). Additional embodiments include approaches to treat and/or prevent HIV infection, wherein an afflicted patient or a person at risk for contracting HIV is provided an amount of modified glycinamide (e.g., alpha-hydroxyglycinamide, α-peroxyglycinamide dimer, diglycinamide ether or alpha-methoxyglycinamide) sufficient to inhibit the replication of HIV. As above, in some embodiments, the compound or a pharmaceutical containing the compound is provided to a subject in need of an agent that inhibits HIV replication and, in other embodiments, the affect on HIV replication is measured (e.g., by measuring a reduction in the viral lode or marker thereof, such as p24 accumulation or reverse transcriptase activity).

### BRIEF DESCRIPTION OF THE DRAWINGS

FIG. 1 shows the structures of glycylprolylglycinamide (GPG-NH₂), sarcosylpyrolylglycinamide (SAR-PG-NH₂), cyclic pyrroglutaminylprolylglycinamide (PyrQPG-NH₂), glutaminylprolylglycinamide (QPG-NH₂), and glycinamide (G-NH₂).

FIG. 2 shows the CD26 activity in human T-lymphocytes (CEM, C8166, Molt4/C8, MT-4) and PBMC suspensions (panel A) or in several different serum (human (HS), murine (MS), bovine (BS) (panel B)) as a function of time. The substrate was glycylprolyl-p-nitroanilide (GP-pNA). Enzyme activity was measured by absorption at 400 nm.

FIG. 3 shows the purified CD26-mediated conversion of unlabeled GPG-NH₂ to GP-OH and G-NH₂. The detection was performed by mass spectrometry.

FIG. 4 shows the conversion of radiolabeled [¹⁴C]GPG-NH₂ to [¹⁴C]G-NH₂ by bovine serum (BS) at 5% in phosphate buffered saline (PBS), Human serum (HS) at 5% in PBS, and CEM cell suspensions (10⁶ cells).

FIG. 5 shows the inhibitory affect of the CD26-specific inhibitor IlePyr on the dipeptidylpeptidase activity of CD26 in 5% bovine serum (BS) in PBS and 10⁶ CEM cell suspensions in PBS using GP-pNA as the substrate.

FIG. 6 shows the effect of the CD26 inhibitor IlePyr on the anti-HIV-1 activity of GPG-NH2 and G-NH2 in CEM cell cultures.

FIG. 7 shows the results of an analysis of several lots of human sera and fetal bovine sera for their ability to convert G-NH₂ to modified G-NH₂ (Metabolite X).

FIG. 8 shows the results of an analysis of different animal sera for their ability to convert G-NH₂ to modified G-NH₂ (Metabolite X).

FIG. 9 shows the results of a competition assay, wherein the ability of different concentrations of glycine, L-serine-NH₂, L-alanine-NH₂, or GPG-NH₂ to inhibit the conversion of G-NH₂ to modified G-NH₂ (Metabolite X) were evaluated.

FIG. 10 shows the results of an analysis of different fractions of fetal bovine serum, obtained by size exclusion chromatography, to convert G-NH₂ to modified G-NH₂ (Metabolite X).

FIG. 11 illustrates the results of a reverse transcriptase (RT) activity assay, wherein enzymatically prepared alpha-hydroxyglycinamide (Metabolite λ or Met-X) inhibited the replication of HIV in cultures containing boiled fetal calf serum but G-NH₂ did not.

FIG. 12 shows the results of a reverse transcriptase (RT) assay, wherein enzymatically prepared alpha-hydroxyglycinamide (Metabolite X or Met-X) that had been dialysed five times inhibited the replication of HIV in cultures containing boiled fetal calf serum.

FIG. 13 shows the results of a reverse transcriptase (RT) assay, wherein the antiretroviral activity (IC₅₀) of various concentrations of enzymatically prepared alpha-hydroxyglycinamide (Metabolite X or Met-X) were analysed.

FIG. 14 shows the results of an HIV infectivity assay (in fetal calf serum) that monitored the accumulation of p24, wherein enzymatically prepared alpha-hydroxyglycinamide (Metabolite X or Met-X) inhibited HIV as effectively as GPG-NH₂.

FIG. 15 shows the results of an HIV infectivity assay (in fetal calf serum) that monitored the accumulation of p24, wherein synthetically prepared alpha-hydroxyglycinamide (AlphaHGA) was observed to inhibit HIV as effectively as GPG-NH₂.

FIG. 16 shows the results of an HIV infectivity assay (in fetal calf serum (panel A) and human serum (panel B)) that monitored the accumulation of p24, wherein enzymatically prepared alpha-hydroxyglycinamide (Metabolite X or Met-X) and synthetically prepared alpha-hydroxyglycinamide (AlphaHGA) inhibited HIV as effectively as G-NH₂ in fetal calf serum (panel A) but only enzymatically prepared alpha-hydroxyglycinamide (Metabolite X or Met-X) and synthetically prepared alpha-hydroxyglycinamide (AlphaHGA) were able to inhibit HIV replication in human serum (panel B).

FIG. 17 shows the results of a reverse transcriptase (RT) assay (in fetal calf serum), wherein the antiretroviral activity of G-NH₂, freshly diluted synthetically prepared alpha-hydroxyglycinamide (AlphaHGA), and synthetically prepared alpha-hydroxyglycinamide, which had been incubated at 37° C. for three days (AlphaHGA 37), was compared.

### DETAILED DESCRIPTION OF THE INVENTION

It has been discovered that some tripeptide amides and glycinamide are prodrugs that are metabolized into compounds that inhibit the replication of HIV. These antiviral agents are highly selective inhibitors in cell culture (e.g., GPG-NH₂ and glycinamide or “G-NH₂,” inhibit HIV replication in CEM cell cultures to an equal extent (50% effective concentration: ˜30 μM)). The focus of research in this area has been on the conversion of tripeptide amides to glycinamide (G-NH₂) since G-NH₂ also inhibits the replication of HIV. (See U.S. patent application Ser. No. 10/235,158, herein expressly incorporated by reference in its entirety). It is now known that the lymphocyte surface glycoprotein marker CD26 efficiently converts GPG-NH₂ to G-NH₂ releasing the dipeptide GP-OH and that this cleavage is required for GPG-NH₂ to exert its antiretroviral activity.

It has also been discovered that G-NH₂ is itself a prodrug that is metabolized to one or more compounds (e.g., cyclic, charged, or uncharged forms of glycinamide) that inhibit the replication of HIV. These metabolites that are derived from G-NH2 are referred to as “modified glycinamide,” “glycinamide derivatives,” or “Metabolite X.” Mass spectrometry and nuclear magnetic resonance (NMR) spectrometry analysis of the modified glycinamide peak fraction isolated after chromatographic separation revealed that it contained o-hydroxyglycinamide (“AlphaHGA” or (C₂H₆N₂O₂) or (C₂H₇ClN₂O₂)). Both α-hydroxyglycinamide and α-methoxyglycinamide were prepared by organic synthesis. It was found that enzymatically prepared alpha-hydroxyglycinamide (Metabolite X) and synthetically prepared alpha-hydroxyglycinamide (AlphaHGA) effectively inhibit HIV in human serum. The formulation of pharmaceuticals and medicaments containing these modified glycinamides is straightforward and the use of these compounds to inhibit replication of HIV in subjects in need thereof is provided herein. The section below describes the discovery that CD26 converts GPG-NH2 to G-NH2 in greater detail.

CD26 Mediates the Conversion of GPG-NH2 to G-NH2

The lymphocyte surface glycoprotein CD26 has been originally described as a T-cell activation/differentiation marker. (See Fox et al., J. Immunol., 132:1250-1256 (1984)). CD26 is abundantly expressed on the target cells of HIV (i.e., lymphocytic CEM, Molt, C8166 and MT-4, and peripheral blood mononuclear cells) and is also present in serum from bovine, murine and human origin. It is a membrane-associated peptidase identical to dipeptidyl-peptidase IV (DPP IV, EC3.4.14.5) and has a high (but not exclusive) selectivity for peptides that contain a proline or alanine as the penultimate amino acid at the N-terminus. (See Yaron and Naider, Biochem. Mol. Biol, 28:31-81 (1993); De Meester et al., Immunol. Today, 20:367-375 (1999) and Mentlein, Regul Pept., 85:9-24 (1999)). It is not only expressed on a variety of leukocyte cell subsets, but also on several types of epithelial, endothelial and fibroblast cells. (Id.). A soluble form of CD26 also exists. It lacks the transmembrane regions and intracellular tail and is detected in plasma and cerebrospinal fluids at low amounts. (See Yaron and Naider, Biochem. Mol. Biol., 28:31-81 (1993); De Meester et al., Immunol Today, 20:367-375 (1999)).

Several cytokines, hematopoietic growth factors, hormones and neuropeptides contain a X-Pro or X-Ala motif at their N-terminus. (See De Meester et al., Immunol. Today, 20:367-375 (1999)). The presence of a proline near the N-terminus serves as a structural protection against non-specific proteolytic degradation. (See Vanhoof et al., FASEB J., 9:736-744 (1995)). In particular, relatively small peptides may serve as natural substrates (e.g., the chemokines RANTES (68 amino acids) and SDF-1α (68 amino acids), and the glucagon/VIP (Vasoactive Intestinal Protein) family peptides such as GIP (42 amino acids) and GLP-2 (33 amino acids)). (See De Meester et al., Immunol. Today, 20:367-375 (1999)). In some cases, the peptides are very short (e.g., the neuropeptides endomorphin 2 (4 amino acids) and substrate P (11 amino acids)). Enterostatin, consisting of only 5 amino acids is also found to be a substrate for CD26.

Interestingly, in certain cases, CD26 was shown to alter the biological functions of natural peptides after it cleaved off a dipeptide part from the N-terminal part of the molecule. (Oravecz et al., J. Exp. Med., 186:1865-1872 (1997); Proost et al., J. Biol. Chem., 273:7222-7227 (1998)). Indeed, truncated RANTES (3-68) was found to have a markedly increased anti-HIV-1 activity compared with intact RANTES (see Schols et al., Antiviral Res., 39:175-187 (1998)); whereas N-terminal processing SDF-1α by CD26 significantly diminished its anti-HIV-1 potency. (See Ohtsuki et al., FEBS Lett., 431:236-240 (1998); Proost et al., FEBS Lett., 432:73-76 (1998)). Also, it was recently shown that CD26 regulates SDF-1α-mediated chemotaxis of human cord blood CD34⁺ progenitor cells. (See Christopherson et al., J. Immunol., 169:7000-7008 (2002)).

The tripeptide glycylprolylglycinamide (GPG-NH₂) has been found to inhibit HIV replication at non-toxic concentrations. (See e.g., U.S. Pat. No. 5,627,035, herein expressly incorporated by reference in its entirety) but its association with CD26 has not been made until this disclosure. Glycylprolylglycinamide blocks a wide variety of HIV-1 laboratory strains and clinical isolates within a range of 2-40 μM. Since there exist two GPG motifs in HIV p24 and one GPG motif in the V3 loop of the viral envelope protein gp120 initial research had been focussed on these viral proteins as potential targets for this novel tripeptide derivative. (See Su, Ph.D. thesis at the Karolinska Institute (ISBN 91-628-4326-5), Stockholm, Sweden (2000) and Su et al., AIDS Res. Human Retrovir., 16:37-48 (2000), herein expressly incorporated by reference in its entirety).

Although an increased SDS-PAGE mobility of gp160/120 was observed at high concentrations of GPG-NH₂, it was found that GPG-NH₂ did not affect an early event in the infection cycle of HIV. (See Su et al., J. Hum. Virol., 4:8-15 (2001)). In addition, binding of GPG-NH₂ with the p24 protein has been demonstrated and an increased number of misassembled core structures of virus particles was observed in GPG-NH₂-treated HIV-1-infected cells. (See Hoglund et al., Antimicrob. Agents Chemother., 46:3597-3605 (2002)). Also, viral capsid (p24) formation was found to be disturbed in the presence of the drug. (See Hoglund et al., Antimicrob. Agents Chemother., 46:3597-3605 (2002)). It became clear that GPG-NH₂ inhibited replication of HIV by a novel mechanism.

Given the presence of a proline residue in the middle (equivalent to the penultimate amino acid at the amino terminus) of the GPG-NH₂ peptide molecule, it was thought that GPG-NH₂ can be a substrate for CD26/dipeptidylpeptidase TV and that CD26 enzymatic activity can modulate the antiretroviral activity of the compound. Accordingly experiments were conducted to determine whether CD26/dipeptidylpeptidase IV could convert GPG-NH₂ to G-NH₂ and, indeed, it was discovered that CD26 selectively and efficiently cleaved GPG-NH₂ after the proline residue to release the dipeptide GP-OH and G-NH₂. Moreover, it was also demonstrated that this cleavage was required for GPG-NH₂ to exert its antiretroviral activity. The example below describes these findings in greater detail.

### Example 1

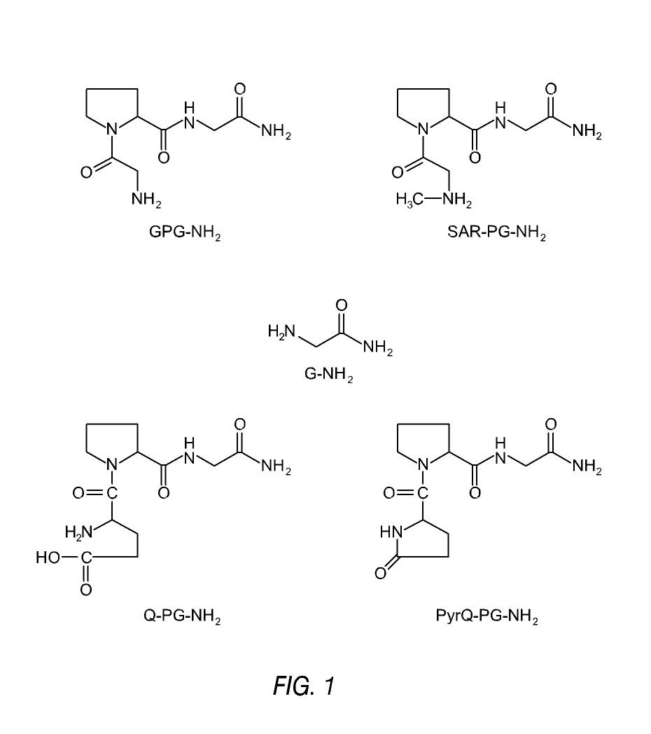

In initial experiments, several HIV-1 and HIV-2 strains were evaluated for their sensitivity to the inhibitory activity of GPG-NH₂, G-NH₂ and related compounds. (See TABLE 1 and FIG. 1). Glycylprolylglycinamide (GPG-NH₂), glutaminylprolylglycinamide (Q-PG-NH₂), sarcosinylprolylglycinamide (Sar-PG-NH₂) and glycinamide (G-NH-₂) were provided by TRIPEP AB (Huddinge, Sweden); whereas, Pyrroglutaminylprolylglycinamine (PyrQ-PG-NH₂) was synthesized at the Rega Institute. Human T-lymphocytic CEM cells were obtained from the American Type culture Collection (Rockville, Md.) and cultured in RPMI-1640 medium (Gibco, Paisley, Scotland supplemented with 10% fetal bovine serum (FBS) (BioWittaker Europe, Verviers, Belgium), 2 mM L-glutamine (Gibco) and 0.075 M NaHCO₃ (Gibco). HIV-1(III_{B}) was obtained from Dr. R. C. Gallo and Dr. M. Popovic (at that time at the National Cancer Institute, NIH, Bethesda, Md.). HIV-1 (NL4.3) was from the National Institute of Allergy and Infectious Disease AIDS Reagent Program (Bethesda, Md.). The HIV-2 isolates ROD and EHO were provided by Dr. L. Montagnier (Pasteur Institute, Paris, France).

Human T-lymphocytic CEM cells (4.5×10⁵ cells per ml) were suspended in fresh cell culture medium and infected with HIV-1 (III_{B} and NL4.3) or HIV-2 (ROD or EHO) at 100 CCID₅₀ (1 CCID₅₀ being the virus dose infective for 50% of the cell cultures) per ml of cell suspension. Then, 100 μl of the infected cell suspension were transferred to microplate wells, mixed with 100 μl of appropriate (freshly prepared) dilutions of the test compounds (i.e., at final concentrations of 2000, 400, 80, 16, 3.2 and 0.62 μM), and were further incubated at 37° C. After 4 to 5 days, giant cell formation was recorded microscopically in the CEM cell cultures. The 50% effective concentration (EC₅₀) corresponded to the compound concentrations required to prevent syncytium formation in the virus-infected CEM cell cultures by 50%.

TABLE 1
Inhibitory activity of tripeptide derivatives against several virus
strains in CEM cell cultures

| | EC₅₀^{a} (μM) | | | | |
|---|---|---|---|---|---|
| | HIV-1 | | HIV-2 | | |
| Compound | III_{B} | NL3.4 | ROD | EHO | CEM |
| GPG-NH₂ | 35 ± 8.7 | 50 ± 0.0 | 30 ± 10 | 42 ± 14 | >2000 |
| G-NH₂ | 32 ± 7.6 | 45 ± 7.1 | 35 ± 8.7 | 37 ± 5.8 | >2000 |
| PyrQ-PG-NH₂ | >2000 | >2000 | >2000 | >2000 | >2000 |
| SAR-PG-NH₂ | 31 ± 4.9 | 49 | 35 ± 9.8 | 56 | >1500 |
| Q-PG-NH₂ | 86 | 265 | 89 | 82 | >1500 |

^{a}50% Effective concentration, or compound concentration required to inhibit HIV-reduced syncytia formation in T-lymphocytic CEM cell cultures

Interestingly, both GPG-NH₂ and G-NH₂ were equally effective in suppressing virus replication on a molar basis, regardless the nature of the virus used in the antiviral assays. Their EC₅₀ (50% effective concentration) ranked between 30 and 50 μM in CEM cell cultures. Both compounds did not show cytotoxicity at concentrations as high as 1500 to 2000 μM. Sar-PG-NH₂ and Q-PG-NH₂ were also inhibitory to HIV replication, although to a lower extent as GPG-NH₂. A novel tripeptide (PyrQ-PG-NH₂) derivative was synthesized containing G-NH₂ at its carboxy terminal end but a cyclic pyrroglutamine at its amino terminal end. In contrast with GPG-NH₂ and the other tripeptide amide derivatives, PyrQ-PG-NH₂ was found to be ineffective at inhibiting HIV replication in cell culture

Next, it was confirmed that CD26 dipeptidylpeptidase activity could be detected in purified CD26 and bovine, murine and human serum and with human lymphocytic or peripheral blood mononuclear cell suspensions. CD26 enzyme activity was recorded by conversion of the synthetic substrate glycylprolyl p-nitroanilide (GP-pNA) to glycylproline (GP-OH) and p-nitroaniline (pNA), a yellow dye, whose formation could be monitored by an increase of the absorption at 400 nm. Approximately, two hundred microliters of purified CD26 (1 milliUnit/ml) in phosphate buffered saline (PBS), or human, murine or bovine serum (5% in PBS) or 106 human lymphocytic CEM, C8166, Molt4/C8, MT-4 or peripheral blood mononuclear cell suspensions in PBS were added to 200 μl-microtiter plate wells after which the substrate for measuring the CD26 enzymatic activity (glycylprolyl-parα-nitroanilide) (GP-pNA) at 3 mM final concentration was added. Glycylprolyl-p-nitroanilide (GP-pNA) and glycylphenylalaninyl-p-nitroanilide (GF-pNA) were obtained from Sigma Chemicals (St. Louis, Mo.). The release of p-nitro-aniline (pNA) was monitored at 37° C. in function of time by measuring the amount of (yellow-colored) parα-nitro aniline (pNA) released from GlyPro-pNA. The pNA release was recorded by the increase of absorption [optical density (OD) at 400 nm] in a Spectramax microplate spectrometer (Molecular Devices, Sunnyvale, Calif.). Under the experimental conditions, the reaction proceeded linearly for at least 60 min. The OD₄₀₀ values of blank reaction mixtures (lacking the CD26 enzyme, serum or cells) were subtracted from the obtained OD₄₀₀ values to represent the real increase of OD₄₀₀ value as a measurement of the enzyme activity.

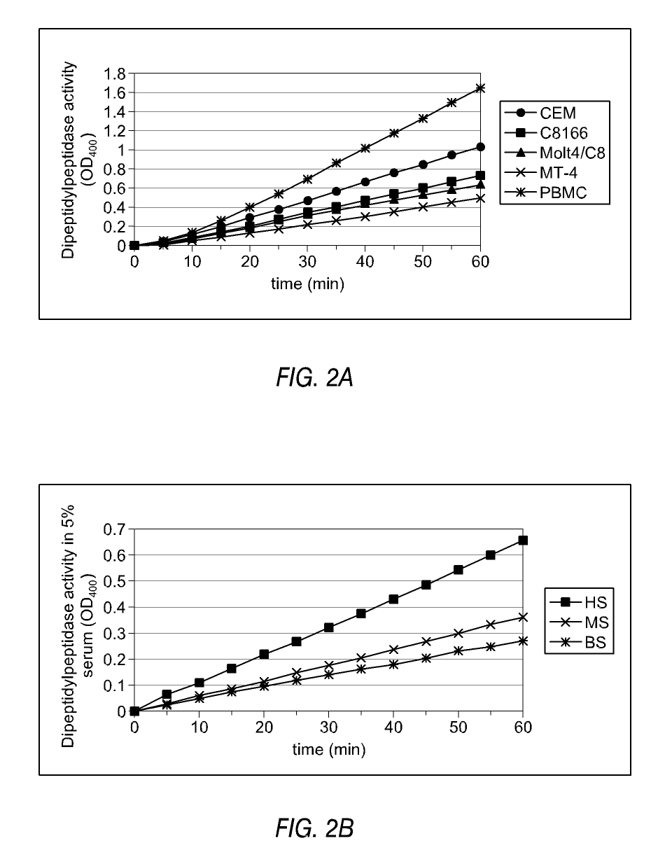

It was found that GP-pNA was only converted by CD26 and not by the action of other dipeptidyl/peptidases since the addition of a specific inhibitor of CD26 to the cell suspensions virtually completely blocked the release of p-nitroaniline from the synthetic substrate GP-pNA (infra). All lymphocytic cell suspensions (CEM, C8166, MT-4, Molt4/C8) and also PBMC at which GP-pNA had been administered efficiently converted GP-pNA to p-nitroaniline in a time-dependent fashion. (See FIG. 2A). The CD26 activity was highest in CEM cell suspensions and lowest in the MT-4 cell suspensions. Also, fetal bovine and murine serum and in particular human serum efficiently released p-nitroaniline from GP-pNA (See FIG. 2B). Thus, both human T-lymphocytic cell suspensions and serum display a prominent CD26/dipeptidylpeptidase enzyme activity. Once it was determined that CD26 activity could be efficiently monitored, experiments were conducted to determine if CD26 could convert GPG-NH₂ to G-NH₂.

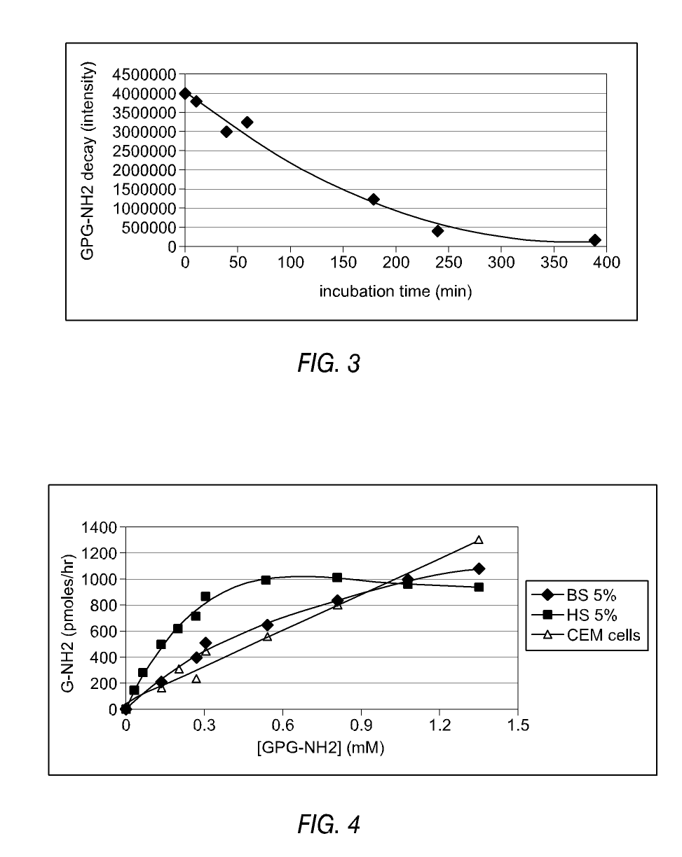

In a sample, approximately, 100 μM GPG-NH₂ was exposed to 25 units/l of purified CD26 and the mixture was incubated for up to 400 minutes at room temperature. The lymphocyte surface glycoprotein CD26/dipeptidylpeptidase IV was purified as described before. (See De Meester, J. Immunol. Methods, 189:99-105 (1996), herein expressly incorporated by reference in its entirety). At different time points, an aliquot of the reaction mixture was withdrawn and analyzed on an electrospray ion trap mass spectrometer (Esquire, Bruker, Bremen, Germany). The appearance of the dipeptide GP-OH upon release from the amino terminal end of the GPG-NH₂ molecule, as well as, the disappearance of intact GPG-NH₂ from the reaction mixture was determined and monitored by electrospray ion trap mass spectometric analysis at different time points. (See FIG. 3). Under these experimental conditions, CD26 released GP-OH in a time-dependent manner from GPG-NH₂, and virtually completely converted GPG-NH₂ to GP-OH and G-NH₂ within 4 to 6 hrs of the reaction. In contrast, CD26 was unable to release G-NH₂ from PyrroQ-PG-NH₂.

Next, the conversion of radiolabeled [¹⁴C]GPG-NH₂ to [¹⁴C]G-NH₂ by purified CD26, fetal bovine serum (FBS), human serum (HS) and CEM cell suspensions was analyzed. Radiolabeled [¹⁴C]GPG-NH₂ (radiospecificity: 58 mCi/mmol), in which the radiolabeled carbon is located in the main chain carbon of the glycine at the carboxylic acid end of the tripeptide, and [¹⁴C]G-NH₂ (radiospecificity: 56 mCi/mmol) in which carbon-2 was radiolabeled were synthesized by Amersham Pharmacia Biotech (Buckinghamshire, England). A variety of these [¹⁴C]GPG-NH₂ concentrations were exposed to purified CD26, FBS, HS and CEM cell suspensions and the conversion to G-NH₂ was analyzed.

In one set of experiments, for example, five-ml CEM cell cultures (5×10⁵ cells/ml) were exposed to 20 μM [¹⁴C]GPG-NH₂ for 24 hrs. Then, the cells were centrifuged for 10 min at 1,200 rpm, washed, and the cell pellet was treated with 60% ice-cold methanol for 10 min. The methanol cell extract was centrifuged for 10 min at 15,000 rpm, after which the supernatant was injected on a cation exchange Partisphere-SCX column (Whattman) to separate GPG-NH₂ from G-NH₂. The following gradient was used: 0-15 min: isocratic buffer A (7 mM sodium phosphate, pH 3.5); 15-40 min linear gradient from buffer A to buffer B (250 mM sodium phosphate, pH 3.5); 40-45 min linear gradient from buffer B to buffer A; 45-55 min: isocratic buffer A. The retention time of [¹⁴C]GPG-NH₂ and [¹⁴C]G-NH₂ under these elution conditions were 26-28 min and 14-16 min, respectively.

In another set of experiments, after one hour of exposure, disappearance of intact [¹⁴C]GPG-NH₂ was determined by HPLC analysis, as described above, using a cation-exchange Partisphere SCX column and a sodium phosphate buffer gradient at pH 3.5. GPG-NH₂ was well-separated from G-NH₂ (retention times: 25-27 min and 15-17 min, respectively). The Kₘ value of CD26-catalyzed conversion of GPG-NH₂ to G-NH₂ was calculated to be 0.183 mM. The estimated Kₘ values of GPG-NH₂ for dipeptidylpeptidase activity associated with HS and FBS were 0.45 and 1.4 mM, respectively, as derived from the GPG-NH₂ disappearance curves depicted in FIG. 4. The GPG-NH₂ conversion by the CEM cell suspensions proceeded linearly up to 1.5 mM. Only at higher GPG-NH₂ concentrations (e.g., 3 and 5.4 mM), did the conversion curve for the CEM cell suspensions start to level-off slightly.

Next, the inhibitory effect of L-isoleucinepyrrolidine (IlePyr) on CD26 was analyzed. Isoleucinepyrrolidine (IlePyr) has recently been reported to be a relatively potent and selective inhibitor of purified CD26-associated dipeptidylpeptidase activity. (See De Meester, J. Immunol. Methods, 189:99-105 (1996)). All enzyme activity assays were performed in 96-well microtiter plates (Falcon, Becton Dickinson, Franklin Lakes, N.J.). To each well were added 5 μl purified CD26 in PBS (final concentration of 0.2 milliUnits/200 μl-well), 10 μl fetal bovine serum (BS) (final concentration: 5% in PBS; preheated at 56° C. for 30 min), or one million CEM cells in PBS, 5 μl of an appropriate concentration of the IlePyr inhibitor solution in PBS (500 and 200 μM) and PBS to reach a total volume of 150 μl. The reaction was started by the addition of 50 μl substrate GP-pNA at 4 mg/ml (final concentration in the 200 μl reaction mixture: 1 mg/ml or 3 mM) and carried out at 37° C. The 50% inhibitory concentration of IlePyr against dipeptidylpeptidase activity associated with CD26, BS and CEM cell suspensions was defined as the compound concentration required to inhibit the enzyme-catalyzed hydrolysis of GP-pNA to pNA and GP-OH by 50%.

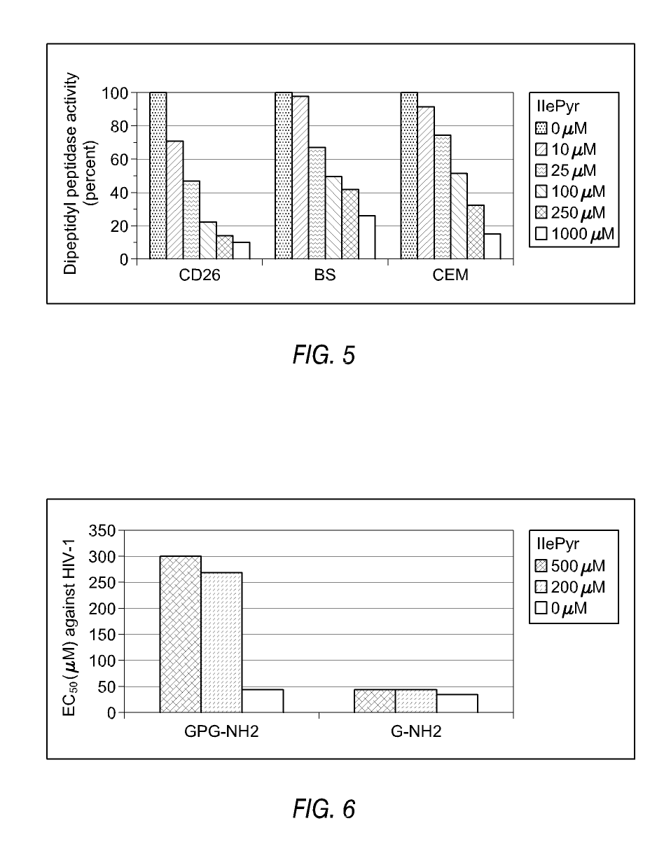

In initial experiments, CD26 inhibition in CEM cell suspensions (in fetal bovine serum) subjected to IlePyr using GP-pNA as the substrate was analyzed. Purified CD26 was included as a positive control. (See FIG. 5). The inhibitor IlePyr dose-dependently prevented release of p-nitroaniline from GP-NA exposed to CEM cell suspensions as well as to fetal bovine serum at a 50% inhibitory concentration (IC₅₀) of 110 and 99 μM, respectively. Purified CD26 was inhibited at an IC₅₀ value of 22 μM. Thus, the 50% inhibitory concentration (IC₅₀) value of the inhibitor IlePyr exposed to serum and CEM cell suspensions was ˜5-fold higher than the inhibitor concentrations required to inhibit purified CD26 by 50%.

Then, experiments were conducted to determine if the antiretroviral activity observed with GPG-NH₂ was associated with the CD26-catalyzed release of G-NH₂ from the tripeptide derivative. HIV-1-infected CEM cell cultures were exposed to different concentrations of GPG-NH₂ in the presence of non-toxic concentrations of IlePyr (500 μM and 200 μM). Similar combinations of G-NH₂ with IlePyr were included in this study. In these experiments, the CD26-specific inhibitor L-isoleucinepyrrolidine (IlePyr), was added to each cell culture microplate prior to the addition of the test compounds and the virus-infected cells.

In contrast with G-NH₂, which fully preserved its anti-HIV activity in CEM cell cultures in the presence of 200 and 500 μM of IlePyr (EC₅₀₋35-43 μM), GPG-NH₂ markedly lost its inhibitory activity against virus-induced cytopathicity in the presence of the specific CD26 inhibitor. (See FIG. 6). The highest inhibitor concentration (500 μM) was slightly more efficient in reversing the anti-HIV-1 activity of the tripeptide GPO-NH₂ than the lower (200 μM) inhibitor concentration. A similar result was observed for Sar-GP-NH₂, another tripeptide amide derivative that is also endowed with antiretroviral activity in cell culture.

The results presented this example, demonstrate that GPG-NH₂ requires hydrolysis to release glycinamide before it is able to exert its anti-HIV activity in cell culture. The data also provide evidence that the release of G-NH₂ from GPG-NH₂ is induced by the enzymatic activity of the lymphocyte surface glycoprotein activation/differentiation marker CD26. The formation of G-NH₂ from GPG-NH₂ was conducted with purified CD26, human T-lymphocyte cell suspensions and human and bovine serum. Moreover, the pronounced antiviral activity of Q-PG-NH₂, the complete lack of antiviral activity of PyrQ-PG-NH₂ (that is resistant to enzymatic attack by CD26) and the loss of antiviral efficacy of GPG-NH₂ and Sar-GP-NH₂ in the presence of a specific inhibitor of CD26 provide strong evidence that GPG-NH₂ acts as an efficient prodrug of G-NH₂ and that CD26-catalyzes the conversion of GPG-NH₂ to G-NH₂.

Accordingly, it was discovered that the lymphocyte surface glycoprotein CD26, which is a membrane associated dipeptidyl peptidase, is the enzyme responsible for metabolizing GPG-NH₂, QPG-NH₁₂, and sarcosylprolylglycinamide (SAR-PG-NH₂) to G-NH₂, for example. More evidence that CD26 was responsible for metabolizing peptide amides into a form that inhibits the replication of HIV was obtained from experiments that employed the selective CD26 inhibitor L-isoleucinepyrrolidine (IlePyr), wherein a significant reduction in the anti-HIV activity of GPG-NH₂ and SAR-PG-NH₂ was observed. The IlePyr inhibitor had no affect on the ability of G-NH₂ to inhibit replication of HIV, however. Thus, X-Pro-glycinamide-containing peptide amides are antiretroviral prodrugs or precursors that are metabolized by the lymphocyte surface glycoprotein CD26 to G-NH₂. The next section describes the discovery that glycinamide inhibits replication of HIV in greater detail.

Glycinamide Inhibits the Replication of HIV

Initially, it was determined that G-NH₂ efficiently inhibits the replication of HIV but compounds that are similar in structure do not. HIV-1 (III_{B})-infected CEM cell cultures were incubated with various concentrations of G-NH₂ or various concentrations of a compound that has a structure similar to G-NH₂ and the inhibition of HIV replication was evaluated using standard procedures. These experiments are described in the next example.

### Example 2

Human T-lymphocytic CEM cells (approx. 4.5×10⁵ cells/ml) were suspended in fresh medium and were infected with HIV-1 (III_{B}) at approx. 100CCID₅₀ per ml of cell suspension (1CCID₅₀ being the virus dose infective for 50% of the cell cultures). Then, 100 μl of the infected cell suspension was transferred to individual wells of a microtiter plate (100 μl/well) and was mixed with 100 μl of freshly diluted test compound (2000, 400, 80, 16, 3.2, or 0.62 μM). Subsequently, the mixtures were incubated at 37° C. After 4 to 5 days of incubation, giant cell formation was recorded microscopically in the CEM cultures. The 50% effective concentration (EC₅₀) corresponded to the concentrations of the compounds required to prevent syncytium formation in the virus-infected CEM cell cultures by 50%.

The results of these experiments are shown in TABLE 2. Glycinamide was found to be the only compound that appreciably inhibited HIV replication in the cell culture. The EC₅₀ for G-NH₂ was approximately 21.3 μM, whereas the other compounds tested showed no inhibition of HIV. These results confirmed that G-NH₂ has a particular structure that inhibits HIV replication.

TABLE 2
Inhibitory activity of compounds against HIV-1
(III_{B}) in CEM cell cultures

| | EC₅₀ (μM)^{a} |
|---|---|
| Glycinamide | 21.3 ± 16.3 |
| Glycin-thioamide | >500 |
| Cyclic glycin-thioamide | >500 |
| L-Alaninamide | >500 |
| L-Leucinamide | >500 |
| L-Isoleucinamide | >500 |
| L-Valinamide | >500 |
| L-Lysinamide | >500 |
| L-Asparaginamide | >500 |
| L-Val β-naphthylamide | >100 |
| Ala-Pro-Gly-Trp-amide | >500 |
| DL-Leucinamide | >500 |
| DL-Tryptophanamide | >500 |
| L-Tyrosinamide | >500 |
| D-Asparagine | >500 |
| L-Phenylalaninamide | >500 |
| L-Methioninamide | >500 |
| L-Threoninamide | >500 |
| L-Argininamide | >500 |
| L-Tryptophanamide | >200 |
| L-Prolinamide | >1000 |
| L-Asparaginamide | >1000 |
| DL-Phenylalaninamide | >1000 |
| D-Leucine | >1000 |
| Sarcosinamide | >1000 |
| L-Serinamide | >1000 |
| L-Alanine | >500 |
| L-Leucine | >500 |
| L-Proline | >500 |
| Glycine | >500 |
| 1,3-diaminoaceton | >1000 |
| Ethylene diamine | >1000 |
| 1,4-diamino-2-butanone | — |
| 1,3-diamino-2-hydroxypropane | >1000 |
| DL-2,3-diaminopropionic acid | >1000 |
| Glycine methylamide | >500 |

^{a}50% effective concentration

Subsequent analysis revealed that G-NH₂ was a specific inhibitor of HIV. The cytotoxicity and antiviral activity of various concentrations of G-NH₂ and GPG-NH₂ were evaluated in cell cultures that were infected with various types of viruses. Conventional host cell culture, viral infection, and infectivity analysis for each different type of cell and virus were followed. Compounds that were known to inhibit replication of the particular types of viruses analyzed were used as controls.

TABLES 3-5 show the results of these experiments. The data show that G-NH₂ and GPG-NH₂ were ineffective at inhibiting the replication of Herpes simplex virus-1 (KOS), Herpes simplex virus-2 (G), Herpes simplex virus-1 TK⁻ KOS ACV^{r}, Vaccinia virus, Vesicular stomatis virus, Coxsackie virus B4, Respiratory syncytial virus, Parainfluenzα-3 virus, Reovirus-1, Sindbis virus, and Punta Toro virus. These results confirmed that G-NH₂ and GPG-NH₂ are selective inhibitors of HIV.

TABLE 3
Cytotoxicity and antiviral activity of compounds in HEL cell cultures

| | | Minimum inhibitory concentration^{b} | | | | |
|---|---|---|---|---|---|---|
| | | | | | | Herpes |
| | Minimum | Herpes | Herpes | | | simplex |
| | Cytotoxic | simplex | simplex | | Vesicular | virus-1 |
| | Concentration^{a} | virus-1 | virus-2 | Vaccinia | stomatitis | TK⁻ KOS |
| Compound | (μg/ml) | (KOS) | (G) | virus | virus | ACV^{r} |
| G-NH₂ (μM) | >2000 | >2000 | >2000 | >2000 | >2000 | >2000 |
| GPG-NH₂ | >400 | >400 | >400 | >400 | >400 | >400 |
| (μM) | | | | | | |
| BVDU | >400 | 0.0256 | >400 | 0.64 | 400 | 400 |
| (μg/ml) | | | | | | |
| Ribavirin | >400 | 48 | >400 | 240 | >400 | 80 |
| (μg/ml) | | | | | | |
| ACG (μg/ml) | >400 | 0.0768 | 0.0768 | >400 | >400 | 9.6 |
| DHPG | >100 | 0.0038 | 0.0192 | 60 | >400 | 0.48 |
| (μg/ml) | | | | | | |

^{a}Required to cause a microscopically detectable alteration of normal cell morphology.
^{b}Required to reduce virus-induced cytopathogenicity by 50%.

TABLE 4
Cytotoxicity and antiviral activity of compounds in HeLa cell cultures

| | Minimum | Minimum inhibitory concentration^{b} | | |
|---|---|---|---|---|
| | cytotoxic | Vesicular | | Respiratory |
| | concentration^{a} | stomatitis | Coxsackie | syncytial |
| Compound | (μg/ml) | virus | virus B4 | virus |
| G-NH₂ (μM) | >2000 | >2000 | >2000 | >2000 |
| GPG-NH₂ | >400 | >400 | >400 | >400 |
| (μM) | | | | |
| Brivudin | ≧400 | >400 | >400 | >400 |
| (μg/ml) | | | | |
| (S)-DHPA | >400 | 240 | >400 | >400 |
| (μg/ml) | | | | |
| Ribavirin | >400 | 9.6 | 48 | 16 |
| (μg/ml) | | | | |

^{a}Required to cause a microscopically detectable alteration of normal cell morphology.
^{b}Required to reduce virus-induced cytopathogenicity by 50%.

TABLE 5
Cytotoxicity and antiviral activity of compounds in Vero cell cultures

| | Minimum | Minimum inhibitory concentration^{b} | | | | |
|---|---|---|---|---|---|---|
| | cytotoxic | | | | | Punta |
| | concentration^{a} | Parainfluenza-3 | | Sindbis | Coxsackie | Toro |
| Compound | (μg/ml) | virus | Reovirus-1 | virus | virus B4 | virus |
| G-NH₂ | >2000 | >2000 | >2000 | >2000 | >2000 | >2000 |
| (μM) | | | | | | |
| GPG-NH₂ | >400 | >400 | >400 | >400 | >400 | >400 |
| (μM) | | | | | | |
| BVDU | >400 | >400 | >400 | >400 | >400 | >400 |
| (μg/ml) | | | | | | |
| (S)-DHPA | >400 | 240 | 80 | >400 | >400 | >400 |
| (μg/ml) | | | | | | |
| Ribavirin | >400 | 48 | 16 | >400 | >400 | 48 |
| (μg/ml) | | | | | | |

^{a}Required to cause a microscopically detectable alteration of normal cell morphology.
^{b}Required to reduce a virus-induced cytopathogenicity by 50%.

It has also been discovered that G-NH₂ is itself a prodrug or precursor that is metabolized by an enzyme or cofactor(s) present in the plasma and sera of some animals to one or more compounds (e.g., cyclic, charged, or uncharged forms of glycinamide) that inhibit the replication of HIV. The section below describes this discovery in greater detail.

Cofactor(s) Present in the Plasma and Sera of Some Animals Converts G-NH₂ to a Metabolite that Inhibits HIV.

Evidence is provided herein that at least one cofactor present in the serum and plasma of some animals metabolizes G-NH₂ to an active form (“modified glycinamide” or Metabolite X), which is transported into cells and inhibits the replication of HIV. Accordingly, G-NH₂ is a precursor or prodrug for an antiretroviral compound and G-NH₂ can be formulated for administration with said cofactor or a material containing said cofactor. Chromatographic methods were used to isolate this cofactor. This cofactor can be purified, cloned, and sequenced using the approaches described herein and conventional techniques in molecular biology. Accordingly, some embodiments include a pharmaceutical or nutriceutical preparation containing G-NH₂ or a compound that metabolizes to G-NH₂ (e.g., GPG-NH₂) formulated in a mixture or administered in conjunction (before or after administration of G-NH₂) with a material that converts G-NH₂ to Metabolite X (e.g., pig serum, plasma, or milk, horse serum, plasma, or milk, bovine serum, plasma, or milk in purified, enriched, or isolated form).

The active form of G-NH₂ (modified glycinamide or Metabolite X) is readily produced by incubation of G-NH₂ in certain serums or plasma and the modified glycinamide is easily isolated by the chromatographic methods described infra. Throughout this disclosure, glycinamide metabolites (the antiretrovirally active forms of glycinamide) are collectively referred to as “modified glycinamide,” “modified G-NH₂,” or “fast peak glycinamide.” Examples of modified G-NH₂ include, but are not limited to α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, and salts and/or derivatives of these compounds. Mass spectrometry and nuclear magnetic resonance (NMR) spectrometry analysis of the modified glycinamide peak fraction isolated after chromatographic separation revealed that it contained α-hydroxyglycinamide. The compound α-peroxyglycinamide dimer (NH₂-gly-O—O-gly-NH₂) may be more stable than α-hydroxyglycinamide and both α-hydroxyglycinamide and α-methoxyglycinamide have been prepared by organic synthesis. Those of skill in the art can readily prepare other modified glycinamide compounds using the procedures described herein and other available synthetic approaches. (See e.g., JP 5097789A2 to Hayakawa et al., entitled “Alpha-hydroxyglycinamide Derivative and its Preparation,” filed Oct. 3, 1991, herein expressly incorporated by reference in its entirety). HIV infectivity studies conducted in the presence of synthetically or enzymatically produced AlphaHGA α-hydroxyglycinamide) revealed that the compound effectively inhibited HIV replication in human serum.

Formulation of the modified G-NH₂ into pharmaceuticals and medicaments, whether the modified G-NH₂ is synthetically produced or produced enzymatically by incubation of G-NH₂ in serum, is straightforward. Accordingly, antiretroviral pharmaceuticals and medicaments can be prepared by providing a modified glycinamide compound (e.g., a compound provided by formulas A, B, C, D, E, F, G, H, or I) or a pharmaceutically acceptable salt thereof in either enantiomer (L or D) or both or either isomer (R or S) or both. Preferred compounds for formulation into an antiretroviral pharmaceutical or medicament include, for example, α-hydroxyglycinamide (formula C), α-peroxyglycinamide dimer (formula E), diglycinamide ether (formula F), and alpha-methoxyglycinamide, or pharmaceutically acceptable salts thereof in either enantiomer (L or D) or both or either isomer (R or S) or both. The antiretroviral pharmaceuticals and medicaments describe herein can be provided in unit dosage form (e.g., tablets, capsules, gelcaps, liquid doses, injectable doses, transdermal or intranasal doses) and can contain, in addition to the modified glycinamide compound, a pharmaceutically acceptable carrier or exipient. Containers comprising said pharmaceuticals and medicaments (e.g., sterile vials, septum sealed vials, bottles, jars, syringes, atomizers, swabs) whether in bulk or in individual doses are also embodiments and, preferably, said formulations are prepared according to certified good manufacturing processes (GMP) (e.g., suitable for or accepted by a governmental regulatory body, such as the Federal Drug Administration (FDA)) and said containers comprise a label or other indicia that reflects approval of said formulation from said governmental regulatory body. Nutriceuticals containing said compounds with or without structure-function indicia are also embodiments, however.

Some embodiments are a preparation for the inhibition of HIV that consists of or is enriched with a modified glycinamide compound (e.g., pharmaceuticals and medicaments for the inhibition of HIV, which consist of, consist essentially of, or comprise, a modified glycinamide compound in an isolated, purified, or synthetic form in an amount that inhibits replication of the virus.) Preferred embodiments include a pharmaceutical or medicament that consists of, consists essentially of, or comprises α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, or derivatives of these compounds.

As used herein, “enriched” means that the concentration of the material is up to 1000 times its natural concentration (for example), advantageously 0.01%, by weight, preferably at least about 0.1% by weight. Enriched preparations from about 0.5%, 1%, 5%, 10%, and 20% by weight are also contemplated. The term “isolated” requires that the material be removed from its original environment (e.g., the natural environment if it is naturally occurring). The term “purified” does not require absolute purity; rather, it is intended as a relative definition. Isolated proteins can be conventionally purified by chromatography and/or gel electrophoresis. Purification of starting material or natural material to at least one order of magnitude, preferably two or three orders, and more preferably four or five orders of magnitude is expressly contemplated.

The following example describes an approach that was used to purify commercially obtained glycinamide. Aspects of this approach were used to purify metabolites of glycinamide produced after incubation in various animal serum, as described infra.

### Example 3

It was observed that when unpurified preparations of [¹⁴C]G-NH₂ were separated by cation exchange high performance liquid chromatography (HPLC) two populations of G-NH₂ were resolved. (See TABLE 6). Crude preparations of radiolabeled G-NH₂ and radiolabeled GPG-NH₂ were separated by HPLC using a cation exchange column (e.g., Partisphere SCX-Whattman). The following gradient was used: 0-15 minutes (isocratic Buffer A composed of 5 mM ammonium phosphate, pH 3.5); 15-40 minutes linear gradient from Buffer A to Buffer B (composed of 250 mM ammonium phosphate, pH 3.5); 40-45 minutes Buffer B; 45-55 minutes linear gradient to Buffer A; and 55-60 minutes isocratic Buffer A to equilibrate the column for the next run.

By this separation approach, the majority of crude [¹⁴C]GPG-NH₂ typically eluted in 26-28 minutes (fractions 26-28), however, trace amounts of radiolabeled compounds eluted in 20-22 minutes (fractions 20-22), 15-17 minutes (fractions 15-17), and 2-3 minutes (fractions 2-3). Approximately 89% of the crude [¹⁴C]G-NH₂ typically eluted in 15-17 minutes (fractions 15-17) but approximately 11% of the crude [¹⁴C]G-NH₂ eluted in 2-3 minutes (fractions 2-3). Trace amounts of crude [¹⁴C]G-NH₂ were also detected in fractions 20-22 and fractions 5-6.

Slight alterations in the buffers and the gradient led to slight shifts in the time of elution of the compounds but, in all preparations, two main populations of glycinamide were detected, a first population that quickly eluted from the column (referred to as the fast peak, fraction 2-3 or fraction 3-4, or impurity in radiolabeled G-NH₂, or modified G-NH₂) and a second population that strongly bound to the column (referred to as the slow peak, fraction 13-14 or fraction 15-17 or G-NH₂). For example, another protocol to isolate modified G-NH₂ used also Buffer A (5 mM ammoniumphosphate pH 3.5) and Buffer B (250 mM ammoniumphosphate pH 3.5). The gradient used with these buffers was as follows: 10 minutes Buffer A; linear gradient to Buffer B for 6 minutes; 2 minutes at Buffer B; then linear gradient to Buffer A for 6 minutes; and equilibration in Buffer A for 6 minutes. By this approach, as well, the G-NH₂, and impurity in radiolabeled G-NH₂ eluted at 10-11 minutes and 2-3 minutes, respectively.

TABLE 6
Purity of [¹⁴C]radiolabeled stock of GPG-NH₂ and G-NH₂

| | Fraction Number on HPLC (cation exchange) | | | | | |
|---|---|---|---|---|---|---|
| Drug | 2-3 | 5-6 | 15-17 | 20-22 | 26-28 | Total |
| G-NH₂ | 53,000 | 1,700 | 435,000 | 1,300 | — | 490,000 |
| | (11%) | (<0.5%) | (89%) | (<0.5%) | | (100%) |
| GPG-NH₂ | 5,100 | — | 700 | 10,600 | 339,000 | 355,400 |
| | (1.5%) | | (<0.5%) | (3%) | (95%) | (100%) |

In this example, an approach to purify commercially obtained G-NH₂ is provided. A modification of this approach has been used to purify modified glycinamide, as described infra. It should be understood that many different cation exchange columns are available for these procedures and many different buffers and gradients can be used. Given the disclosure herein, one of skill in the art can rapidly adapt a particular type of cation exchange column, FPLC or HPLC, buffer, or gradient to isolate modified G-NH₂ (Metabolite X). That is, modifications of the procedures described above are within the skill in the art and are equivalent to the methods described herein.

As discussed in the sections that follow, it was discovered that modified G-NH₂ (fractions 2-3) can be made from unmodified G-NH₂ (fractions 15-17) by incubating unmodified G-NH₂ in various serums or plasma. Modified G-NH₂ that is made in this manner (enzymatically prepared) can then be isolated using one of the approaches above. Using conventional techniques in structure analysis, it was determined that the modified G-NH₂ isolated by the chromatographic procedure above comprised α-hydroxyglycinamide.

Initially, it was observed that if cell culture medium containing fetal bovine serum was heated for 30 minutes at 95° C., the ability of G-NH₂ to inhibit the replication of HIV was lost. In some experiments, human T-lymphocytic CEM cells (approx. 4.5×10⁵ cells/ml) were suspended in fresh medium and were infected with HIV-1 (III_{B}) at approx. 100CCID₅₀ per ml of cell suspension. Subsequently, the infected cells were provided various concentrations of G-NH₂ that had been dissolved in serum (10% fetal bovine serum in PBS) containing RPMI-1640 medium or G-NH₂ that had been dissolved in heat inactivated serum (10% fetal bovine serum in PBS that had been heated to 95° C. for 30 minutes) containing RPMI-1640 medium. The cell resuspensions were then incubated at 37° C. and, after 4 to 5 days, HIV replication was evaluated. It was discovered that the G-NH₂ that had been incubated in heat inactivated serum containing medium had lost its ability to inhibit the replication of HIV. These results provided strong evidence that a heat labile protein present in serum metabolized G-NH₂ to a modified G-NH₂ form that inhibited replication of HIV.

Following the discovery that a heat labile cofactor(s), present in fetal calf serum, could convert G-NH₂ to a antiretrovirally-active form of glycinamide, experiments were conducted to determine if this cofactor(s) was present in human serum and sera from other animals. The following example describes these experiments in greater detail.

### Example 4

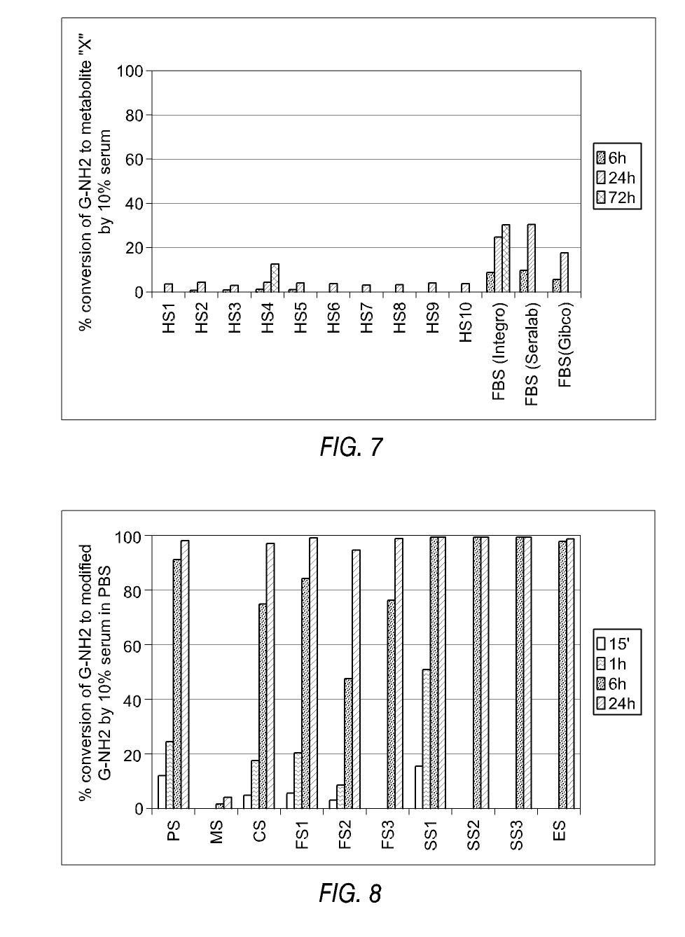

Several lots of human sera and fetal bovine sera were analyzed for their ability to convert G-NH₂ to modified G-NH₂. Radiolabeled cation exchange HPLC purified G-NH₂ (see EXAMPLE 3) was incubated with the various sera at a 10% final concentration in PBS at 37° C. for 15 minutes and 1, 6, 24, or 72 hours. Subsequently, the amount of radiolabeled modified G-NH₂ was evaluated using the cation exchange HPLC approach described above. The results are shown in FIG. 7. Each of the 10 different human serum samples showed less than 10% conversion of G-NH₂ to modified G-NH₂ after 24 hours of incubation. All of the fetal bovine sera tested showed significant conversion of G-NH₂ to modified G-NH₂ after 6 hours (6-10%) and 24 hours (18-32%) of incubation. The results confirmed that fetal bovine sera contained the cofactor(s) that significantly metabolizes G-NH₂ to modified G-NH₂ but human serum does not.

Next, an evaluation of sera obtained from other animals was analyzed for their ability to convert G-NH₂ to modified G-NH₂. Serum obtained from pigs (PS), mice (MS), dogs (CS), cats (FS), horse (ES), and monkey (SS) was incubated with HPLC purified G-NH₂ and at 15 minutes, 1 hour, 6 hours, and/or 24 hours an aliquot of the mixture was removed and analyzed by cation exchange HPLC, as described above. Approximately a 10% dilution of serum in PBS was used. As shown in FIG. 8, the sera obtained from pigs, dogs, cats, horse, and monkeys rapidly converted G-NH₂ to modified G-NH₂, whereas, the mice serum poorly metabolized G-NH₂. The data showed that although several animals were able to metabolize G-NH₂ to modified G-NH₂, the ability of the cofactor(s) to metabolize G-NH₂ was not evolutionarily conserved in humans and mice.

Several experiments were also performed to better characterize the cofactor(s) found in pig plasma. In one set of experiments, pig plasma was dialyzed (MW cut off 10,000) and the dialysate was evaluated for the ability to convert G-NH₂ to modified G-NH₂. Various concentrations of G-NH₂ were mixed with either 90% pig plasma or 90% dialyzed pig plasma and were incubated for 24 hours at 37° C. Subsequently, aliquots of the mixtures were separated by cation exchange HPLC, as described previously, and the conversion of G-NH₂ to modified G-NH₂ was evaluated. TABLE 7 shows the results of these experiments. The data show that the conversion of G-NH₂ to modified G-NH₂ was almost identical in both the pig plasma and dialyzed pig plasma samples. Saturation of the enzyme activity of cofactor(s) in pig plasma (90% in PBS) occurred between 1,000M and 10,000 μM G-NH₂. These results provided more evidence that the cofactor(s) that metabolizes G-NH₂ to modified G-NH₂ is a protein found in plasma or serum.

TABLE 7
Conversion of G-NH₂ to modified G-NH₂
by dialyzed pig plasma (24 hr)

| | |
|---|---|
| | conversion to modified G-NH₂ |
| Concentration G-NH₂ | (24 hr) (percent conversion) |

| (μM) | Pig plasma^{a} | Dialysed Pig plasma |
|---|---|---|
| 18 | 99.7 | 99.8 |
| 100 | 99.7 | 99.8 |
| 1,000 | 98.7 | 99.8 |
| 10,000 | ~24.5 | 24.7 |

^{a}Plasma: 90% in PBS.

In another set of experiments, the saturation point of the cofactor(s) found in dialyzed pig plasma was more closely scrutinized. Dialyzed pig plasma (90% in PBS) was mixed with concentrations of G-NH₂ between 2,000 μM and 10,000M. Subsequently, the mixtures were incubated at 37° C. for 6 hours and aliquots were separated by cation exchange HPLC, as before. The results shown in TABLE 8 confirmed that the saturation point of the cofactor(s) in pig plasma was near 2,000 μM G-NH₂.

TABLE 8
Conversion of G-NH₂ to modified G-NH₂ by dialyzed
pig plasma^{a} (6 hr)

| Concentration G-NH₂ | | |
|---|---|---|
| (μM) | Percent conversion | μM formation |
| 2,000 | 82.6 | 1,652 |
| 4,000 | 42.1 | 1,684 |
| 6,000 | 24.9 | 1,494 |
| 8,000 | 21.0 | 1,680 |
| 10,000 | 17.0 | 1,700 |

^{a}Plasma: 90% in PBS.

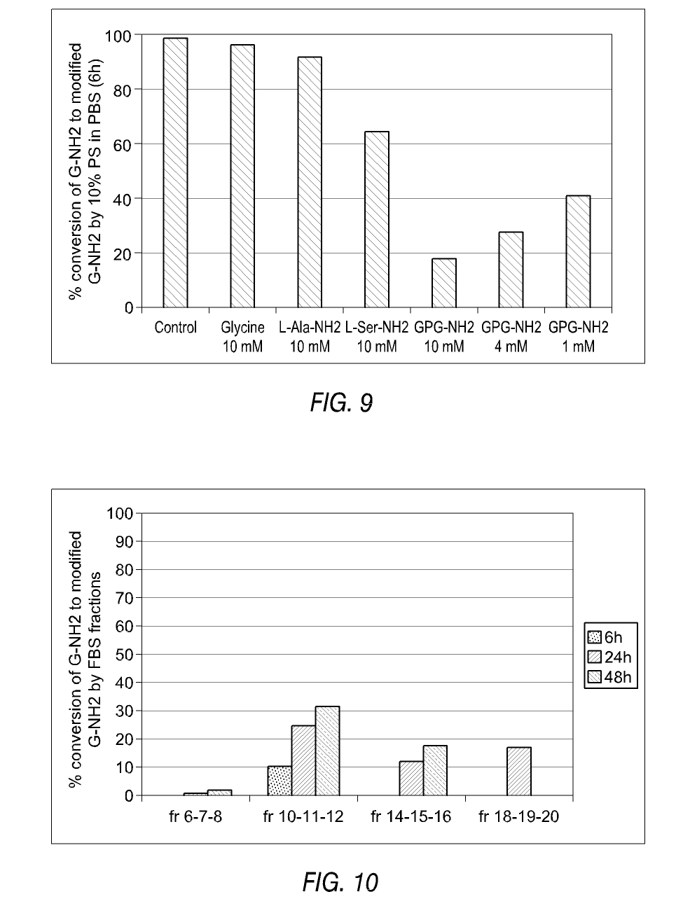

Amino acid competition studies were also employed to determine if the cofactor(s) present in pig serum was specific for G-NH₂. In these experiments, approximately 10% pig serum in PBS was incubated for 6 hours at 37° C. in the presence of 18 μM G-NH₂ and a competitor (10 μM, 40 μM, 100 μM, 400 μM, 1000 μM, 4,000 μM, or 10,000 μM glycine, 10,000 μM L-serine-NH₂, 10,000 μM L-alanine-NH₂, 1000 μM, 4,000 μM, or 10,000 μM GPG-NH₂). A control without competitor was also evaluated. Subsequently, the conversion of G-NH₂ to modified G-NH₂ was analyzed by cation exchange HPLC, as before. The results shown in FIG. 9 provided evidence that the cofactor(s) present in pig serum was specific for G-NH₂ although GPG-NH₂ seemed also to have an inhibitory effect on the G-NH₂ conversion.

Once it had been confirmed that certain sera contained the cofactor(s) that could convert G-NH₂ to modified G-NH₂, experiments were conducted to isolate the cofactor(s). The example below describes these experiments in greater detail.

### Example 5

In a first set of experiments designed to isolate the cofactor(s) that converts G-NH₂ to modified G-NH₂, size exclusion chromatography (Superdex 200) was employed to separate the components present in fetal bovine serum. The separation was for 60 minutes in milli Q water and 30 fractions (0.5 ml/min) were collected. The presence of cofactor(s) in the various fractions was ascertained by incubating an aliquot of the isolated fraction with HPLC purified G-NH₂ followed by an analysis of the presence or absence of modified G-NH₂, as determined by cation exchange HPLC. As shown in FIG. 10, the majority of the cofactor eluted from the size exclusion column in fractions 10-12. Fractions 10-12 were found to efficiently convert G-NH₂ to modified G-NH₂, as determined by monitoring the accumulation of modified G-NH₂ by HPLC cation exchange chromatography, as described previously. Fractions 10-12 were also found to restore the anti-HIV activity of G-NH₂ in heated serum. The activity detected in later fractions may be a result of partially degraded co-factor or cofactor that non-specifically interacted with the resin employed. This data confirmed that the cofactor that converts G-NH₂ to modified G-NH₂ had been isolated. The cofactor can now be purified, sequenced, and cloned using conventional techniques in protein purification and molecular biology.

After incubating the G-NH₂ with serum, the modified G-NH₂ can be isolated from G-NH₂ using cation exchange HPLC, by chromatography (e.g., see EXAMPLE 3), and the anti-HIV activity of purified, modified G-NH₂ (fractions 2-3) and purified G-NH₂ (fractions 15-17) can be compared in a conventional HIV infectivity assay. The effects of modified glycinamide compounds (e.g., α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, and/or derivatives thereof), on HIV replication can also be analysed in this manner.

For example, the EC₅₀ for the purified, modified G-NH₂ (fractions 2-3), purified G-NH₂ (fractions 15-17), α-hydroxyglycinamide, α-peroxyglycinamide dimer (NH₂-gly-O—O-gly-NH₂), diglycinamide ether (NH₂-gly-O-gly-NH₂), α-methoxyglycinamide, and α-ethoxyglycinamide or a derivative thereof is determined using the HIV infectivity assay described previously. Briefly, human T-lymphocytic CEM cells (approx. 4.5×10⁵ cells/ml) are suspended in fresh medium and are infected with HIV-1 (III_{B}) at approx. 100CCID₅₀ per ml of cell suspension. Then, 100 μl of the infected cell suspension is transferred to individual wells of a microtiter plate (100 μl/well) and is mixed with 100 μl of freshly diluted modified G-NH₂ (fraction 2-3), G-NH₂ (fraction 15-17), α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, or a derivative thereof (e.g., 2000, 400, 80, 16, 3.2, and 0.62 μM). Subsequently, the mixtures are incubated at 37° C. After 4 to 5 days, giant cell formation is recorded microscopically in the CEM cultures. The 50% effective concentration (EC₅₀) is then determined.

The results from this set of experiments will show that modified G-NH₂ (fraction 2-3), α-hydroxyglycinamide, α-peroxyglycinamide dimer (NH₂-gly-O—O-gly-NH₂), diglycinamide ether (NH₂-gly-O-gly-NH₂), α-methoxyglycinainde, α-ethoxyglycinamide, or the derivative thereof has a comparable or lower EC₅₀ than G-NH₂ (fraction 15-17). For example, modified G-NH₂, α-hydroxyglycinamide, α-peroxyglycinamide dimer (NH₂-gly-O—O-gly-NH₂), diglycinamide ether (NH₂-gly-β-gly-NH₂), α-methoxyglycinamide, α-ethoxyglycinamide, and the derivative will have an EC₅₀ of approximately 25 μM or less, whereas, G-NH₂ will have an EC₅₀ of approximately 30 μM. These experiments will provide more evidence that G-NH₂ is metabolized to modified G-NH₂, which is the active form of the anti-viral agent.

As another example, the ability of modified G-NH₂ to inhibit the replication of HIV in heat inactivated serum (30 minutes at 95° C.) or human serum-containing medium is compared. Human T-lymphocytes (e.g., approx. 4.5×10⁵ cells/ml of CEM cells) are suspended in fresh medium containing fetal bovine serum and are infected with HIV-1 (III_{B}) at approx. 100CCID₅₀ per ml of cell suspension. Then, the infected cells are washed in PBS and resuspended in medium containing 10% fetal bovine serum that was heated for 30 minutes at 95° C. or human serum. Next, 100 μl of the infected cell suspension is transferred to individual wells of a microtiter plate (100 μl/well) and is mixed with 100 μl of freshly diluted purified, modified G-NH₂ (fraction 2-3), α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, or a derivative thereof, or purified G-NH₂ (fraction 15-17) (e.g., 2000, 400, 80, 16, 3.2, and 0.62 μM). Subsequently, the mixtures are incubated at 37° C. After 4 to 5 days of incubation, giant cell formation is recorded microscopically in the cultures. The 50% effective concentration (EC₅₀) is then determined. The results from this set of experiments will show that the purified, modified G-NH₂ (fraction 2-3), α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, or a derivative thereof efficiently inhibits replication of HIV in the boiled fetal bovine serum or human serum samples, whereas purified G-NH₂ (fraction 15-17) does not. The following example describes experiments that demonstrated that enzymatically prepared o-hydroxyglycinamide (Metabolite X) effectively inhibits the replication of HIV.

### Example 6

Modified glycinamide was enzymatically produced, isolated, and analysed for its ability to inhibit the replication of HIV. Dialysis tubing (3500 kD molecular weight cut-off) was shaken in distilled water with PEST buffer (RPMI with streptomycin and penicillin) for 30 min at room temperature followed by shaking in 2% sodium bicarbonate and 1 mM EDTA for 30 min at 60° C. The tubing was rinsed two times in distilled water with PEST. After that, the tubing was boiled in distilled water with PEST for 5 min. After boiling, the tubing was transferred to a beaker filled with PBS+PEST, and stored at +4° C. until used.

The tubing was used 20 days after boiling. On a sterile bench, the dialysis tubing was washed with sterile and deionised water. Approximately, 10 ml of porcine serum (Promeda corp.) was added to the tubing. The tubing was put in a glass beaker filled with 200 ml PBS-A/PEST (1 ml PEST+1L PBS-A). The beaker was taken out of the sterile bench and placed on an orbital shaker. After 1 h, the PBS-A/PEST was replaced with 200 ml fresh PBS-A=“pre-wash”. The tubing was pre-washed five times with five portions of PBS-A for 1 h as described above. After the pre-wash, the dialysis tubing containing serum, was transferred to a sterile glass bottle filled with 100 ml of sterile filtrated 1 mM glycinamide (Bachem) and a magnetic stirring bar. The bottle containing the glycinamide and serum was incubated on a magnetic stirring plate at 37° C. After approximately 48 h, the dialysis was stopped, the dialysis solution was divided into three portions (10 ml+38 ml+50 ml) and was transferred to labelled glass bottles, which were sealed and frozen at −85° C. A portion of the frozen dialysis solution was then freeze dried.

The freeze-drying system (Vacuum oil (Heto 88900100), Milli-Q water, water purification equipment, Freeze-dryer, and -85° C. freezer) were prepared. Frozen dialysis solution (the 38 ml portion from 1-1) was transferred from the −85° C. freezer to the freeze-drying chamber. The lid was placed over the chamber and the vacuum was turned on. The freeze-drying process was stopped after approximately 72 h. The vacuum was turned off and the glass bottle was removed from the freeze-drying chamber.

Next, freeze-dried product was purified by HPLC. Approximately, 2 L of 0.1M KH₂PO₄ (Merck no. 14873-250/Lot: A397373251) was prepared by weighing 27.22 g KH₂PO₄ and dissolving it in 2 L water (pH˜4.06). The column (Hypersil SCX ion-exchange column 5 um/250×10 mm (ThermoQuest 3-34087/Batch: 5/100/5580) and HPLC-system including software D-7000 HSM) was equilibrated with mobile phase (90% 0.1M KH₂PO₄/10% acetonitrile (Scharlau AC0329/Batch:57048)) for 60 min at 5 ml/min. The UV-detector wavelength was set for 206 nm. The dried dialysis “sample” was dissolved in 2 ml water (19 mM glycine-amide was present at the start of dialysis) and was injected and analysed (RUN-1) with a 10 min isocratic run of mobile phase (see above) at 5 ml/min. The injection volume for RUN 1 was approximately 100 μl.

After calibration, 200 μl of sample was injected nine more times (RUN-2→10) and fractions eluting at 2.5-3.1 min were collected for each run using a TIME-mode collection set for 0.1 min/fraction. Between RUN-8 and 9, 1 L 0.1M KH₂PO₄ was prepared by weighing 13.61 g KH₂PO₄ and dissolving it in 1 L water. The corresponding fractions collected in RUN-2→10, were pooled and were injected over the column (RUN-11→116). In RUN-11→16 each injection contained approximately 100 μl. The fractions were collected between 2.6-2.8 min and were pooled. Approximately, 1.25 mg of modified glycinamide (Metabolite X) was obtained, as determined from the amount of original glycinamide and the area of the collected peaks. The pooled 2.6-2.8 min fractions in 7.5 ml of mobile phase (90% 0.1M KH₂PO₄/10% acetonitrile) were transferred to a labelled glass bottle that was sealed and frozen at 85° C. Additionally, 7.5 ml mobile phase was frozen at 85° C. as a salt control. HPLC-analysis revealed that all detectable glycinamide (retention time˜5.9 min) had been converted to modified glycinamide (2.7 min). After analysis/purification, the column was washed with 40% acetonitrile/water for 31 min at 5 ml/min and the enzymatically prepared modified glycinamide (“Metabolite X”) was freeze-dried using the approach described above.

An HIV infectivity assay was then performed with the enzymatically prepared modified glycinamide (MetX). The lyophilised MetX (1.25 mg) was dissolved in 7.5 ml sterile distilled water (2.24 mM MetX). Approximately, 3.7 ml of 2.24 mM MetX was mixed with 4.8 ml each of normal and boiled RPMI++ (RPMI-medium with 10% FCS and 0.1% PEST). That is, two lots of 8.5 ml of 1 mM MetX were prepared. Then, approximately 3 ml 1 mM MetX was mixed with 3 ml each of normal and boiled RPMI++ (i.e., 2×6 ml of 500 uM MetX). Approximately, 1 ml 500 uM MetX was then mixed with 4 ml each of normal and boiled RPMI++ yielding 2×5 ml of 100 uM MetX. The lyophilised salt control was dissolved and diluted exactly the same as MetX, above. A 1 mM stock solution of unmodified glycinamide was also used to prepare 100 μM glycinamide in normal and boiled RPMI++ (controls) as described for MetX, as well.

H9 cells were counted in three A-squares of a Burke chamber (a mean of 1.2×10⁶ cells/ml, which is 4×10⁶ cells in 3.3 ml). Approximately, 4×10⁶ cells (3.3 ml) were added to two 50 ml tubes. Next, approximately 14.7 ml of normal RPMI++ was added to the first tube and approximately 14.7 ml boiled RPMI++ was added to the second tube (i.e., 18 ml H9 cells+normal/boiled RPMI++). Then approximately 2 ml of virus stock (SF2+H9, day 9:22/3-02 2) was added to each 50 ml tube containing the cells and medium, about 20 ml/tube, and the solutions were mixed. The two virus/cell mixtures were split into two new 50 ml tubes (i.e., four tubes with 10 ml of cell/virus (two tubes with normal RPMI++ and two with boiled RPMI++)). The cell/virus tubes were incubated at 37° C. for 90 min with mixing after 50 min. The infection was stopped by collecting the cells (5 min at 1200 rpm). The cells were then resuspended and transferred to 12 10 ml tubes (0.5×10⁶ cells/tube). That is, six tubes of cells suspended in normal RPMI++ and six tubes of cells suspended in boiled RPMI++. The cells were washed with RPMI (without additives) and collected (5 min at 1500 rpm). The supernatants were discarded and the cells were resuspended in 4.5 ml each of:

Normal RPMI++

Boiled RPMI++

100 μM glycine-amide in normal RPMI++

100 μM glycine-amide in boiled RPMI++

500 μM MetX in normal RPMI++

500 μM MetX in boiled RPMI++

100 μM MetX in normal RPMI++

100 μM MetX in boiled RPMI++

500 μM salt in normal RPMI++

500 μM salt in boiled RPMI++

100 μM salt in normal RPMI++

100 μM salt in boiled RPMI++

Approximately, 0.9 ml/well of each cell suspension (four replicates of each) was added to a 48-well plate as follows:

### PLATE-1:

4 wells with 100 M glycine-amide in normal RPMI++

4 wells with 100 μM glycine-amide in boiled RPMI++

4 wells with 500 μM MetX in normal RPMI++

4 wells with 500 μM MetX in boiled RPMI++

4 wells with 100 μM MetX in normal RPMI++

4 wells with 100 μM MetX in boiled RPMI++

### PLATE-2:

4 wells untreated normal RPMI++

4 wells untreated boiled RPMI++

4 wells “100 μM” salt in normal RPMI++

4 wells “100 μM” salt in boiled RPMI++

4 wells “500 μM” salt in normal RPMI++

4 wells “500 μM” salt in boiled RPMI++

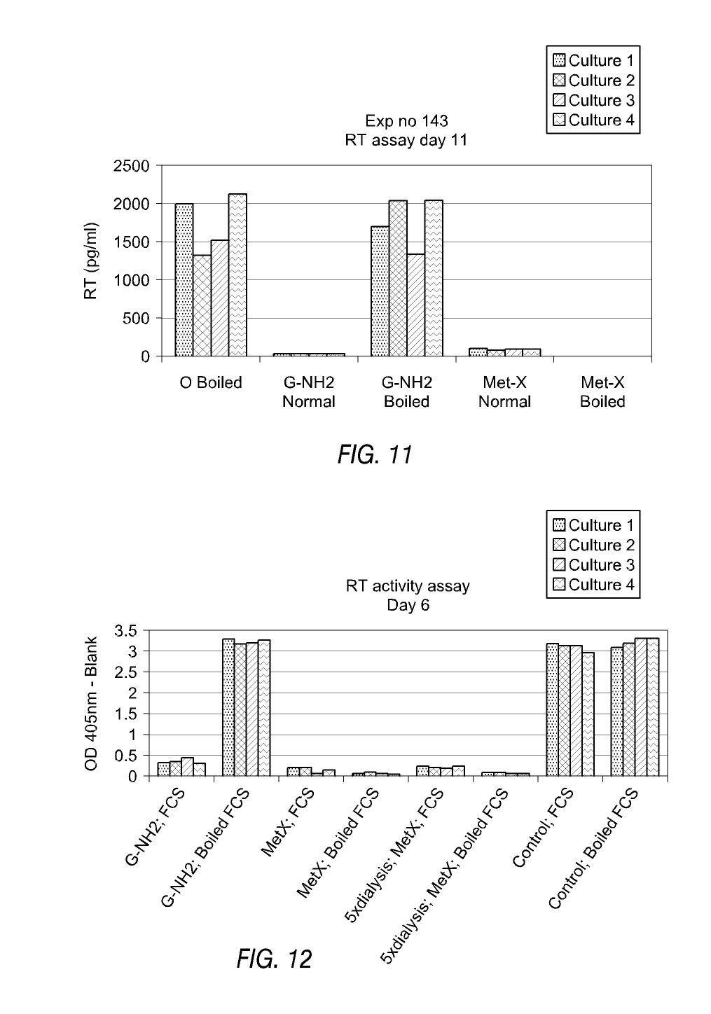

The remaining wells were filled with sterile distilled water. The cell culture plates were incubated at 37° C. and 5% CO₂. After four days the medium was changed, after eight days the medium was changed and the cells were collected. After 11 days, the infection was stopped, the cells were viewed in a 10× magnification microscope and 650 μl of each cell supernatant was collected and frozen at −80° C. for further analysis. After five more days, the supernatants were thawed and used in a conventional reverse transcriptase (RT) activity assay (e.g., Roche AMPLICOR MONITOR™) or a p24 quantification assay (e.g., Abbott Laboratories, Chicago). (See U.S. Pat. No. 6,258,932 and U.S. patent application Ser. No. 10/235,158, both of which are hereby expressly incorporated by reference in its entireties). The results are shown in FIG. 11 and TABLE 9.

TABLE 9

| Sample | Visible syncytia |
|---|---|
| 100 μM MetX in normal RPMI++ | negative |
| 100 μM MetX in boiled RPMI++ | negative |
| 500 μM MetX in normal RPMI++ | negative |
| 500 μM MetX in boiled RPMI++ | negative |
| 100 μM glycinamide in normal RPMI++ control | negative |
| 100 μM glycinamide in boiled RPMI++ control | positive |
| Untreated normal RPMI control | positive |
| Untreated boiled RPMI control | positive |
| 100 μM salt control in normal RPMI++ | positive |
| 100 μM salt control in boiled RPMI++ | positive |
| 500 μM salt control in normal RPMI++ | negative |
| 500 μM″ salt in boiled RPMI++ | negative |

By visual inspection, modified glycinamide (Metabolite X) effectively inhibited replication and/or propagation of HIV in the boiled fetal calf serum but glycinamide did not (TABLE 9). The reverse transcriptase (RT) activity data (FIG. 11) confirmed that modified glycinamide (Met-X or Metabolite X) effectively inhibited replication HIV in the boiled fetal calf serum sample even though G-NH₂ was unable to inhibit replication of HIV under these conditions. That is, the antiviral activity of modified glycinamide (MetX) does not require a cofactor(s) that is present in fetal calf serum but glycinamide does. This data also indicates that the heating of the fetal calf serum denaturated the enzyme (cofactor(s)) that converts glycinamide to modified glycinamide.

In another set of related experiments, the antiretroviral activity of Metabolite X that had been dialysed five times was compared to Metabolite X prepared by the approach above. In brief, HIV infectivity assays were performed with G-NH₂ in fetal calf serum, as above, with the five-times dialysed Metabolite X and the Metabolite X prepared by the approach above. The results of these experiments are shown in FIG. 12. A significant change in the activity of the five-time dialysed alpha-hydroxyglycinamide (Metabolite X), as compared to the standard preparation of the enzymatically produced alpha-hydroxyglycinamide (Metabolite X) was not observed.

The modified glycinamide obtained according to the enzymatic approach described above has been analysed by mass spectroscopy and NMR and the structure analysis revealed alpha-hydroxyglycinamide (“AlphaHGA”). Thus, the experiments in this example have shown that modified glycinamide (alpha-hydroxyglycinamide or Metabolite X) effectively inhibits the replication of HIV in the absence of the cofactor(s) present in fetal calf serum that is required for the antiretroviral activity of G-NH₂. Alpha hydroxyglycinamide (“AlphaHGA”) has also been prepared synthetically and was found to inhibit HIV replication in the absence of the cofactor(s), as described infra.

In more experiments, the 50% inhibitory concentration (IC₅₀) of Metabolite X was analysed in cell cultures containing fetal calf serum. The example below describes these experiments in greater detail.

### Example 7

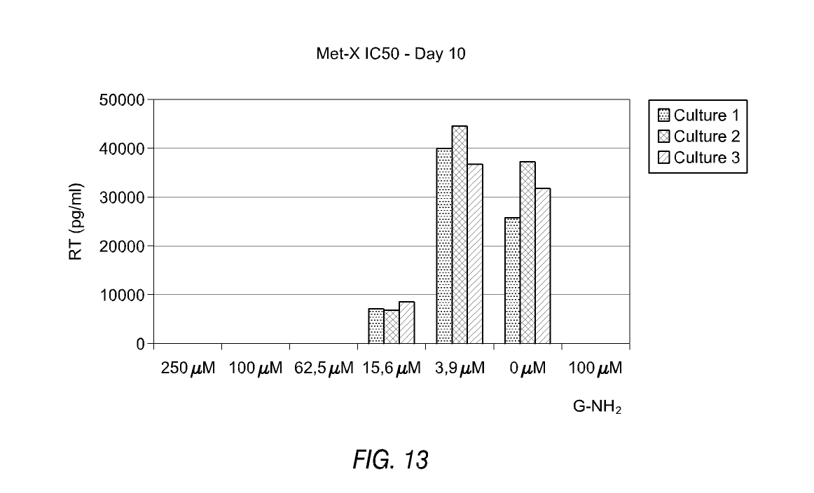

Approximately, 0.1×10⁶H9 cells were infected with 50 TCID₅₀ HIV (SF2 virus) and the infected cells were treated with enzymatically prepared Metabolite X (see EXAMPLE 6) at various concentrations. Fetal bovine serum was included in the assay. The cells were cultured for 10 days (fresh medium was added to the cultures day 7), after which the supernatants were collected and analyzed by a conventional reverse transcriptase (RT) quantification assay. The data is shown in FIG. 13. The results show that effective inhibition of HIV replication occurs at low concentrations of Metabolite X (e.g., between 3.9 μM -15.6 μM) and that when concentrations reach 15.6 μM or higher, the inhibition of HIV replication is virtually complete.

In more experiments, enzymatically prepared modified glycinamide (Metabolite X) was incubated with HIV infected H9 cells (SF2 virus) and the morphology of the treated virus was sent to be analysed by electron microscopy. As a positive control, GPG-NH₂ was used. (See U.S. Pat. No. 6,258,932, herein expressly incorporated by reference in its entirety, for an approach to perform these type of electron microscopy experiments). The example below describes these experiments in greater detail.

### Example 8

By one approach, modified glycinamide (Metabolite X) was enzymatically prepared by the dialysis of purified G-NH₂ against pig serum (see EXAMPLE 6); the modified glycinamide was then used to treat HIV (SF2 virus) infected H9 cells, and the infected cells were sent for analysis by electron microscopy. In brief dialysis tubing (3500 MW cut-off-Spectrum) was loaded with pig serum (Biomedia) and the pig serum was pre-dialyzed against RPMI 1640 buffer four times for one hour each to remove molecules that were less than 3500 daltons. The pre-washed serum was then dialysed against 1 mM purified G-NH₂ in RPMI 1640 at 37° C. for 48 hours. The dialysed buffer containing the modified G-NH₂ (Metabolite X) was then sterile filtered, aliquoted, and frozen, as described in EXAMPLE 6.

Next, a 100 μm Metabolite X or 100 μM GPG-NH₂ concentration was established in four bottles containing (each) approximately 0.5×10⁶H9 cells in 10 ml of RPMI (containing fetal calf serum). The cells in the samples were counted and then centrifuged. The cells were then resuspended in 10 ml of RPMI 1640 (containing fetal calf serum) and either 100 nm Metabolite X or 100 μM GPG-NH₂. Uninfected control and untreated control samples were also included in the experiment. The samples were then incubated overnight at 37° C. at 5% CO₂.

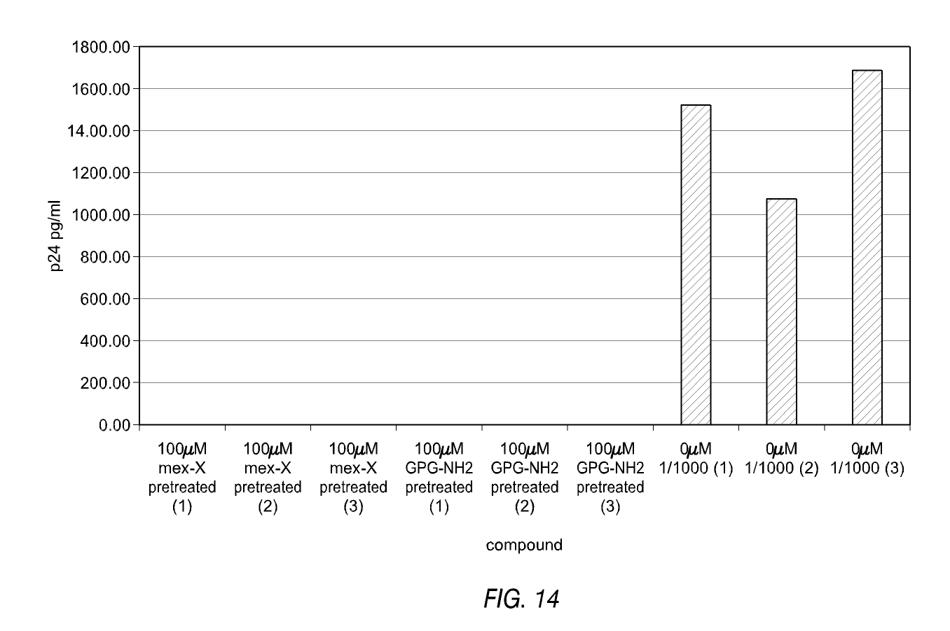

Then, the amount of p24 in the samples was analysed using a conventional p24 detection assay (see U.S. Pat. No. 6,258,932). As shown in FIG. 14, 100 μM modified glycinamide (Metabolite X) or 100 μM GPG-NH₂ effectively inhibited HIV replication in the presence of fetal calf serum; whereas, the untreated control samples showed appreciable HIV replication. These results were confirmed by a conventional reverse transcriptase (RT) activity assay, which showed appreciable amounts of reverse transcriptase activity in the untreated control samples but no reverse transcriptase activity in the samples treated with 100 μM modified glycinamide or 100 μM GPG-NH₂. Having verified that the samples treated with 100 μM modified glycinamide or 100 μM GPG-NH₂ contained virus that had been inhibited, the samples were sent to be analysed by electron microscopy.

By one approach, H9 cells that were infected by SF2 virus can be fixed in 2.5% glutaraldehyde by conventional means. The fixed cells are then postfixed in 1% OsO₄ and are dehydrated, embedded with epoxy resins, and the blocks are allowed to polymerize. Epon sections of virus infected cells are made approximately 60-80 nm thin in order to accommodate the width of the nucleocapsid. The sections are mounted to grids stained with 1.0% uranyl acetate and were analyzed in a Zeiss CEM 902 microscope at an accelerating voltage of 80 kV. The microscope is equipped with a spectrometer to improve image quality and a liquid nitrogen cooling trap is used to reduce beam damage. The grids having sections of control GPG-NH₂ incubated cells and metabolite X incubated cells are examined in several blind studies.

The electron microscopy of untreated HIV particles will show the characteristic conical-shaped nucleocapsid and enclosed uniformly stained RNA that stretched the length of the nucleocapsid; whereas, the cells having HIV-1 particles that are treated with GPG-NH₂ or Metabolite X will show HIV-1 particles having conical-shaped capsid structures that appear to be relatively intact but the RNA was amassed in a ball-like configuration either outside the capsid or at the top (wide-end) of the capsid. Some capsids from the GPG-NH₂ or Metabolite X treated samples may be observed to have misshapen structures with little or no morphology resembling a normal nucleocapsid and the RNA may be either outside the structure or inside the structure at one end.

In still more experiments, the antiretroviral activity of G-NH₂, GPG-NH₂, enzymatically prepared modified glycinamide (Metabolite X), and synthetically prepared modified glycinamide (AlphaHGA) were compared. The example below describes these experiments in greater detail.

### Example 9

HIV infectivity assays were performed in the presence of fetal calf serum, as described in the preceding examples (see EXAMPLES 6-8), however, various concentrations of G-NH₂, GPG-NH₂, and enzymatically prepared modified glycinamide (Metabolite X), and 100 μM synthetically produced modified glycinamide (AlphaHGA) were used. (See TABLE 10). Three replicate samples (“replicates”) of uninfected samples and untreated samples were also included in the experiment as controls. The inhibition of HIV replication was monitored by quantifying the levels of p24 using a conventional detection kit.

TABLE 10

| Peptide | Conc. | | Samples |
|---|---|---|---|
| GPG-NH₂ | 100 | μM | 3 replicates at each |
| | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| G-NH₂ | 100 | μM | 3 replicates at each |
| | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| Met-X (enzymatically prepared by | 100 | μM | 3 replicates at each |
| dialysis) | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| AlphaHGA (synthetically | 100 | μM | 3 replicates |
| produced by Chemilia) | | | |

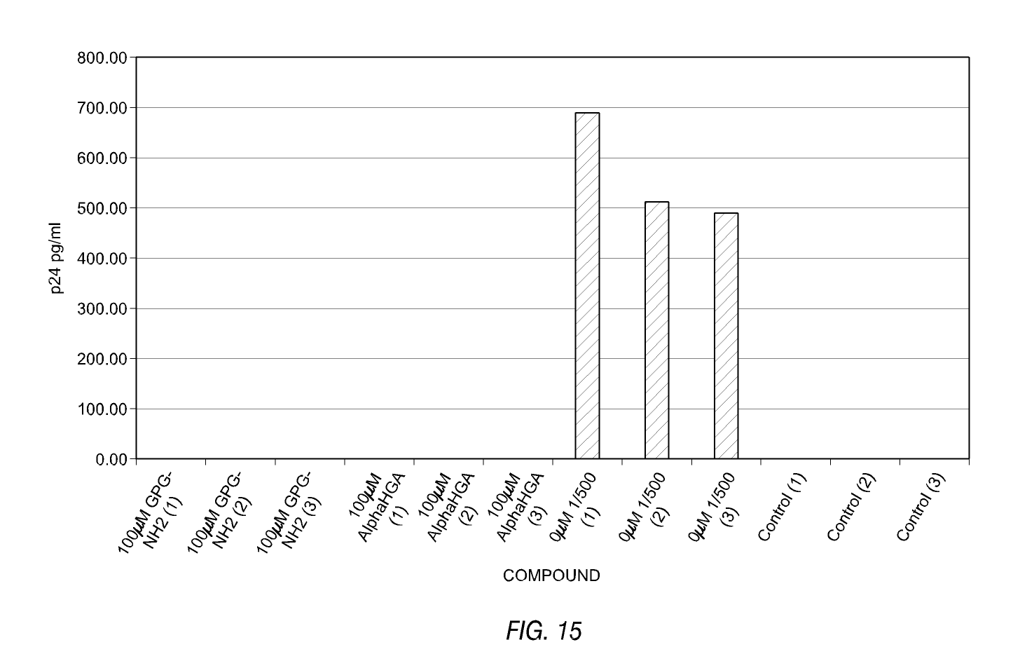

FIG. 15 shows some of the results of these experiments. As shown, on day 11 of the experiment, the synthetically produced alpha-hydroxyglycinamide (AlphaHGA) inhibited HIV replication as effectively as GPG-NH₂ in fetal calf serum-containing media. Similar results were also observed at day 7. This data demonstrate that synthetically produced alpha-hydroxyglycinamide (AlphaHGA) effectively inhibits HIV replication.

In still more experiments, the antiretroviral activity of enzymatically prepared and synthetically prepared alpha hydroxyglycinamide, in the presence of human or fetal calf serum, were compared. The following example describes these experiments in greater detail.

### Example 10

HIV infectivity assays were performed in the presence of human serum or fetal calf serum, as described in the preceding examples (see EXAMPLES 6-8), however, various concentrations of G-NH₂, enzymatically prepared modified glycinamide (Metabolite X), and 100 μM synthetically produced modified glycinamide (AlphaHGA) were used. (See TABLES 11 and 12). Three replicates of uninfected samples and untreated samples were also included in the experiment as controls.

TABLE 11
Human serum

| Peptide | Conc. | Samples |
|---|---|---|
| G-NH₂ | 100 μM | 3 replicates at each |
| | 50 μM | concentration |
| Met-X (enzymatically prepared by | 100 μM | 3 replicates at each |
| dialysis) | 50 μM | concentration |
| Alpha HGA (synthetically | 50 μM | 3 replicates |
| prepared by Chemilia) | | |
| Uninfected control | 0 μM | 3 replicates |
| Infected control | 0 μM | 3 replicates |

TABLE 12
Fetal calf serum

| Peptide | Conc. | Samples |
|---|---|---|
| G-NH₂ | 100 μM | 3 replicates at each |
| | 50 μM | concentration |
| Met-X (enzymatically prepared by | 100 μM | 3 replicates at each |
| dialysis) | 50 μM | concentration |
| Alpha HGA (synthetically | 50 μM | 3 replicates |
| prepared by Chemilia) | | |
| Uninfected control | 0 μM | 3 replicates |
| Infected control | 0 μM | 3 replicates |

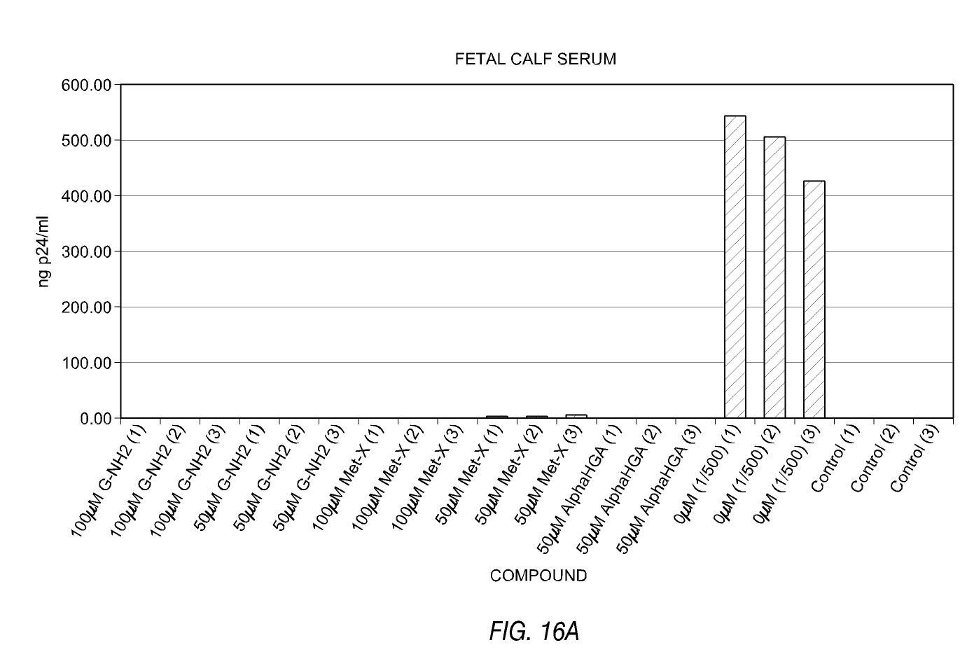

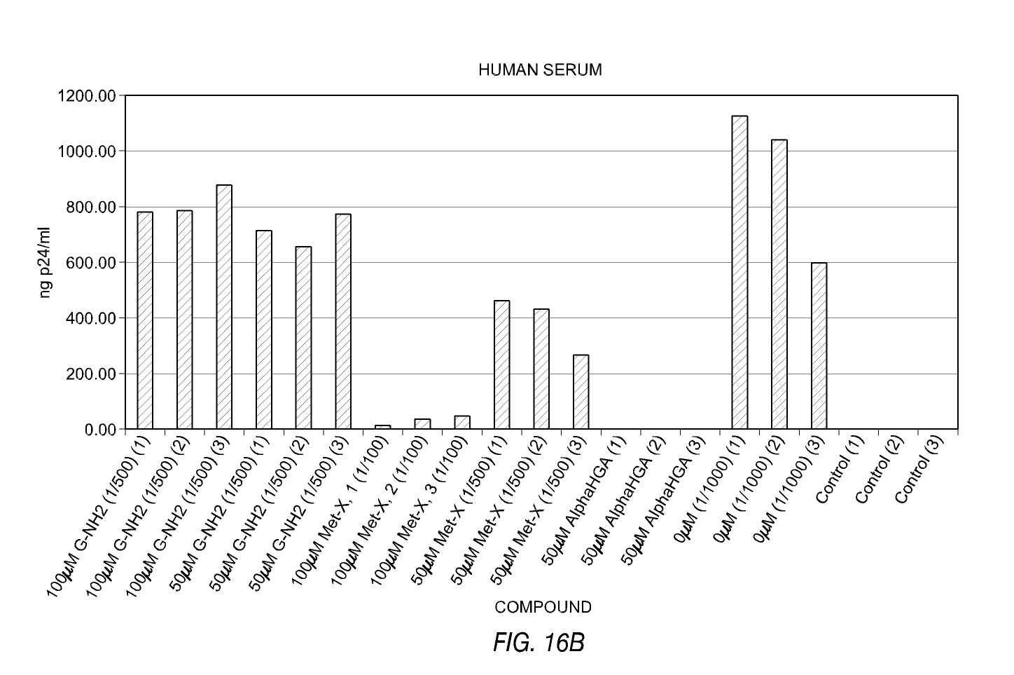

The results of these experiments are provided in TABLES 13 and 14 and in FIGS. 16A and 16B. The data show that on day 12, the enzymatically prepared modified glycinamide (Metabolite X), and the synthetically produced alpha-hydroxyglycinamide (AlphaHGA) inhibited HIV replication as effectively as G-NH₂ in fetal calf serum-containing media; however, only the enzymatically prepared modified glycinamide (Metabolite X), and synthetically produced alpha-hydroxyglycinamide (AlphaHGA) were able to inhibit HIV replication in human serum. That is, G-NH₂ was unable to inhibit HIV replication in human serum but both enzymatically prepared modified glycinamide (Metabolite X), and synthetically produced alpha hydroxyglycinamide (AlphaHGA) were effective inhibitors of HIV replication in human serum. Similar results were observed at day 7. This data provides strong evidence that both enzymatically prepared modified glycinamide (Metabolite X), and synthetically produced alpha hydroxyglycinamide (AlphaHGA) are potent inhibitors of HIV replication in infected humans.

TABLE 13
Fetal Calf serum

| | | | mean | mean OD - | conc p24 |
|---|---|---|---|---|---|
| | OD1 | OD2 | OD | blank | (ng/ml) |
| 100 μM G-NH2 (1) | 0.078 | 0.075 | 0.077 | 0.035 | 0.09 |
| 100 μM G-NH2 (2) | 0.071 | 0.069 | 0.070 | 0.028 | 0.08 |
| 100 μM G-NH2 (3) | 0.077 | 0.071 | 0.074 | 0.032 | 0.09 |
| 50 μM G-NH2 (1) | 0.319 | 0.335 | 0.327 | 0.285 | 0.49 |
| 50 μM G-NH2 (2) | 0.182 | 0.183 | 0.183 | 0.141 | 0.26 |
| 50 μM G-NH2 (3) | 0.105 | 0.103 | 0.104 | 0.062 | 0.14 |
| 100 μM Met-X (1) | 0.193 | 0.343 | 0.268 | 0.226 | 0.40 |
| 100 μM Met-X (2) | 0.081 | 0.107 | 0.094 | 0.052 | 0.12 |
| 100 μM Met-X (3) | 0.144 | 0.152 | 0.148 | 0.106 | 0.21 |
| 50 μM Met-X (1) | 1.105 | 1.089 | 1.097 | 1.055 | 1.71 |
| 50 μM Met-X (2) | 1.895 | 1.887 | 1.891 | 1.849 | 2.98 |
| 50 μM Met-X (3) | 2.351 | 2.230 | 2.291 | 2.249 | 3.61 |
| 50 μM AlphaHGA (1) | 0.183 | 0.185 | 0.184 | 0.142 | 0.26 |
| 50 μM AlphaHGA (2) | 0.232 | 0.216 | 0.224 | 0.182 | 0.33 |
| 50 μM AlphaHGA (3) | 0.147 | 0.139 | 0.143 | 0.101 | 0.20 |
| 0 μM (1/500) (1) | 0.691 | 0.717 | 0.704 | 0.662 | 544.90 |
| 0 μM (1/500) (2) | 0.673 | 0.637 | 0.655 | 0.613 | 505.98 |
| 0 μM (1/500) (3) | 0.544 | 0.568 | 0.556 | 0.514 | 427.33 |
| Control (1) | 0.042 | 0.039 | 0.041 | −0.001 | 0.04 |
| Control (2) | 0.042 | 0.037 | 0.040 | −0.002 | 0.03 |
| Control (3) | 0.046 | 0.045 | 0.046 | 0.004 | 0.04 |

TABLE 14
Human serum

| | | | | | conc |
|---|---|---|---|---|---|
| | | | mean | mean OD - | p24 |
| | OD1 | OD2 | OD | blank | (ng/ml) |
| 100 μM G-NH2 (1/500) (1) | 1.194 | 1.196 | 1.195 | 1.111 | 780.21 |
| 100 μM G-NH2 (1/500) (2) | 1.184 | 1.221 | 1.203 | 1.119 | 785.24 |
| 100 μM G-NH2 (1/500) (3) | 1.315 | 1.362 | 1.339 | 1.255 | 876.34 |
| 50 μM G-NH2 (1/500) (1) | 1.079 | 1.114 | 1.097 | 1.013 | 714.23 |
| 50 μM G-NH2 (1/500) (2) | 0.996 | 1.015 | 1.006 | 0.922 | 653.27 |
| 50 μM G-NH2 (1/500) (3) | 1.176 | 1.194 | 1.185 | 1.101 | 773.51 |
| 100 μM Met-X (1/100) (1) | 0.117 | 0.114 | 0.116 | 0.032 | 11.41 |
| 100 μM Met-X (1/100) (2) | 0.269 | 0.281 | 0.275 | 0.191 | 32.78 |
| 100 μM Met-X (1/100) (3) | 0.377 | 0.378 | 0.378 | 0.294 | 46.52 |
| 50 μM Met-X (1/500) (1) | 0.698 | 0.728 | 0.713 | 0.629 | 457.33 |
| 50 μM Met-X (1/500) (2) | 0.676 | 0.662 | 0.669 | 0.585 | 427.85 |
| 50 μM Met-X (1/500) (3) | 0.418 | 0.422 | 0.420 | 0.336 | 261.05 |
| 50 μM AlphaHGA (1) | 1.546 | 1.546 | 1.546 | 1.462 | 2.03 |
| 50 μM AlphaHGA (2) | 1.183 | 1.219 | 1.201 | 1.117 | 1.57 |
| 50 μM AlphaHGA (3) | 0.665 | 0.679 | 0.672 | 0.588 | 0.86 |
| 0 μM (1/1000) (1) | 0.887 | 0.857 | 0.872 | 0.788 | 1127.68 |
| 0 μM (1/1000) (2) | 0.827 | 0.791 | 0.809 | 0.725 | 1043.27 |
| 0 μM (1/1000) (3) | 0.472 | 0.472 | 0.472 | 0.388 | 591.77 |
| Control (1) | 0.095 | 0.089 | 0.092 | 0.008 | 0.08 |
| Control (2) | 0.091 | 0.089 | 0.090 | 0.006 | 0.08 |
| Control (3) | 0.081 | 0.089 | 0.085 | 0.001 | 0.07 |

In another series of experiments, the stability of synthetically prepared alpha-hydroxyglycinamide (AlphaHGA) to prolonged heating at 37° C. was analysed. Diluted samples of synthesized AlphaHGA (C₂H₇ClN₂O₂), were incubated at 37° C. for periods of time and then the antiretroviral activity of the incubated compound was compared to that of freshly diluted AlphaHGA. These experiments are described in greater detail in the example below.

### Example 11

HIV infectivity assays were performed in the presence of fetal calf serum, as described in the preceding examples (see EXAMPLES 6-8), however, various concentrations of G-NH₂, synthetically produced modified glycinamide (AlphaHGA), and synthetically produced modified glycinamide that had been incubated at 37° C. for three days were used (AlphaHGA 37). (See TABLE 15). Three replicates of uninfected samples and untreated samples were also included in the experiment as controls.

TABLE 15

| Peptide | Conc. | Samples |
|---|---|---|
| αHGA | 32 μM | 3 replicates at each |
| | 16 μM | concentration |
| | 8 μM | |
| | 4 μM | |
| | 2 μM | |
| | 1 μM | |
| | 0.5 μM | |
| αHGA 37 | 32 μM | 3 replicates at each |
| (incubated at 37° C. for three days) | 16 μM | concentration |
| | 8 μM | |
| | 4 μM | |
| | 2 μM | |
| | 1 μM | |
| | 0.5 μM | |
| G-NH₂ | 32 μM | 3 replicates at each |
| | 16 μM | concentration |
| | 8 μM | |
| | 4 μM | |
| | 2 μM | |
| | 1 μM | |
| | 0.5 μM | |

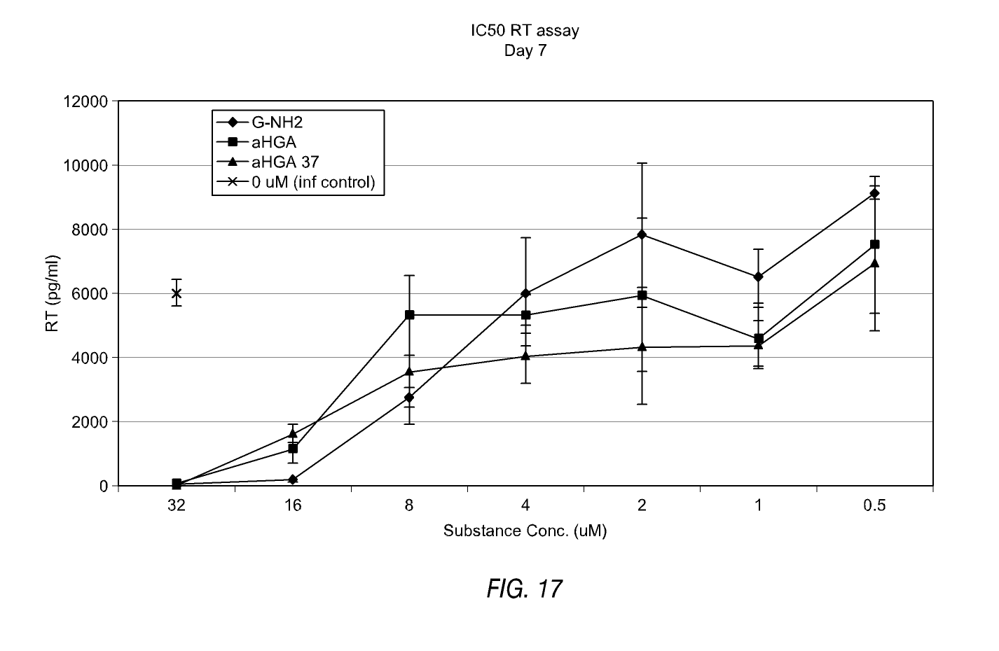

The results of these experiments are shown in FIG. 17 and TABLE 16. FIG. 17 shows a plot of the RT activity detected at day 7. Similar results were obtained when the RT activity was analysed at day 11. The data show that synthetically prepared AlphaHGA is stable to incubation at 37° C. for at least three days. Very little difference in the antiretroviral activity of freshly diluted AlphaHGA and the incubated compound was observed. Further, these data show that appreciable inhibition of HIV replication occurs with synthetic AlphaHGA (whether heat-treated or not) at concentrations above 8 μM, better antiretroviral activity was observed at concentrations above 16 μM, and very efficient inhibition of HIV replication was seen at concentrations above 30 μM. Interestingly, the Metabolite X formed from the conversion of G-NH₂ by the fetal calf serum in the assay (see the data on the G-NH₂ sample) was more active than the synthetically purified AlphaHGA, which provides evidence that one enantiomer and/or isomer of AlphaHGA has more antiretroviral activity than the other.

TABLE 16

| | | | | OD₄₀₅₋₆₂₀ - | | | | |
|---|---|---|---|---|---|---|---|---|
| Compound | Conc. (μM) | | OD₄₀₅₋₆₂₀ | Blank | RT (pg/ml) | StAv | Conc. | mean |
| Control | 0 | | 0.631 | 0.605 | 6318 | 420 | 0 | 6029 |
| | 0 | | 0.622 | 0.596 | 6221 | | | |
| | 0 | | 0.56 | 0.534 | 5547 | | | |
| G-NH2 | 32 | | * | * | * | 2 | 32 | 21 |
| | 32 | | 0.155 | 0.129 | 23 | | | |
| | 32 | | 0.141 | 0.115 | 20 | | | |
| | 16 | | 0.563 | 0.537 | 112 | 40 | 16 | 158 |
| | 16 | | 0.861 | 0.835 | 176 | | | |
| | 16 | | 0.902 | 0.876 | 185 | | | |
| | 8 | | 0.274 | 0.248 | 2438 | 315 | 8 | 2750 |
| | 8 | | 0.302 | 0.276 | 2742 | | | |
| | 8 | | 0.332 | 0.306 | 3068 | | | |
| | 4 | | 0.781 | 0.755 | 7949 | 1682 | 4 | 6029 |
| | 4 | | 0.493 | 0.467 | 4818 | | | |
| | 4 | | 0.539 | 0.513 | 5318 | | | |
| | 2 | | 0.868 | 0.842 | 8895 | 2252 | 2 | 7789 |
| | 2 | | 0.903 | 0.877 | 9275 | | | |
| | 2 | | 0.528 | 0.502 | 5199 | | | |
| | 1 | | 0.563 | 0.537 | 5579 | 838 | 1 | 6514 |
| | 1 | | 0.672 | 0.646 | 6764 | | | |
| | 1 | | 0.712 | 0.686 | 7199 | | | |
| | 0.5 | | 0.871 | 0.845 | 8927 | 205 | 0.5 | 9152 |
| | 0.5 | | 0.896 | 0.87 | 9199 | | | |
| | 0.5 | | 0.908 | 0.882 | 9329 | | | |
| αHGA | 32 | μM | 0.269 | 0.243 | 48 | 25 | 32 | 72 |
| | 32 | μM | 0.373 | 0.347 | 70 | | | |
| | 32 | μM | 0.497 | 0.471 | 97 | | | |
| | 16 | μM | 0.189 | 0.163 | 1514 | 431 | 16 | 1134 |
| | 16 | μM | 0.111 | 0.085 | 666 | | | |
| | 16 | μM | 0.162 | 0.136 | 1221 | | | |
| | 8 | μM | 0.665 | 0.639 | 6688 | 1256 | 8 | 5300 |
| | 8 | μM | 0.507 | 0.481 | 4971 | | | |
| | 8 | μM | 0.44 | 0.414 | 4242 | | | |
| | 4 | μM | 0.541 | 0.515 | 5340 | 615 | 4 | 5315 |
| | 4 | μM | 0.481 | 0.455 | 4688 | | | |
| | 4 | μM | 0.594 | 0.568 | 5916 | | | |
| | 2 | μM | 0.786 | 0.76 | 8003 | 2397 | 2 | 5934 |
| | 2 | μM | 0.647 | 0.621 | 6492 | | | |
| | 2 | μM | 0.354 | 0.328 | 3308 | | | |
| | 1 | μM | 0.564 | 0.538 | 5590 | 945 | 1 | 4594 |
| | 1 | μM | 0.462 | 0.436 | 4482 | | | |
| | 1 | μM | 0.391 | 0.365 | 3710 | | | |
| | 0.5 | μM | 0.692 | 0.666 | 6982 | 2153 | 0.5 | 7539 |
| | 0.5 | μM | 0.962 | 0.936 | 9916 | | | |
| | 0.5 | μM | 0.576 | 0.55 | 5721 | | | |
| αHGA 37 | 32 | μM | 0.198 | 0.172 | 32 | 11 | 32 | 43 |
| | 32 | μM | 0.243 | 0.217 | 42 | | | |
| | 32 | μM | 0.296 | 0.27 | 54 | | | |
| | 16 | μM | 0.171 | 0.145 | 1318 | 282 | 16 | 1641 |
| | 16 | μM | 0.219 | 0.193 | 1840 | | | |
| | 16 | μM | 0.212 | 0.186 | 1764 | | | |
| | 8 | μM | 0.549 | 0.523 | 5427 | 1654 | 8 | 3558 |
| | 8 | μM | 0.322 | 0.296 | 2960 | | | |
| | 8 | μM | 0.26 | 0.234 | 2286 | | | |
| | 4 | μM | 0.33 | 0.304 | 3047 | 909 | 4 | 4050 |
| | 4 | μM | 0.444 | 0.418 | 4286 | | | |
| | 4 | μM | 0.493 | 0.467 | 4818 | | | |
| | 2 | μM | 0.64 | 0.614 | 6416 | 1847 | 2 | 4329 |
| | 2 | μM | 0.317 | 0.291 | 2905 | | | |
| | 2 | μM | 0.387 | 0.361 | 3666 | | | |
| | 1 | μM | 0.512 | 0.486 | 5025 | 713 | 1 | 4420 |
| | 1 | μM | 0.473 | 0.447 | 4601 | | | |
| | 1 | μM | 0.384 | 0.358 | 3634 | | | |
| | 0.5 | μM | 0.891 | 0.865 | 9145 | 2147 | 0.5 | 6978 |
| | 0.5 | μM | 0.496 | 0.47 | 4851 | | | |
| | 0.5 | μM | 0.688 | 0.662 | 6938 | | | |

The section that follows describes the preparation of pharmaceuticals that contain modified glycinamide and the use of these compositions to treat, prevent, and/or inhibit replication of HIV.

Compounds that Inhibit HIV

As discussed above, in addition to G-NH₂ and modified G-NH₂, certain derivatives and metabolites of G-NH₂ inhibit HIV replication and these compounds can be formulated into a medicament or pharmaceutical, which can be used to inhibit HIV replication and treat and/or prevent HIV infection. Some pharmaceuticals or medicaments consist of, consist essentially of, or comprise a compound of formula A:

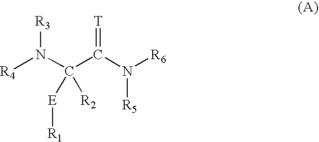

or a pharmaceutically acceptable salt, amide, ester, or prodrug thereof; wherein:

a) E is selected from the group consisting of oxygen, sulfur, and NR₇;

b) T is selected from the group consisting of oxygen, sulfur, and NR₈; and

c) R₁-R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl.

Accordingly, the term “modified G-NH₂ or modified glycinamide compound” includes derivatives and metabolites of glycinamide, such as those of formula A, as described herein, whether enriched or isolated from a cell or synthetically prepared (e.g., α-hydroxyglycinamide, α-peroxyglycinamide dimer (NH₂-gly-O—O-gly-NH₂), α-methoxyglycinamide, α-ethoxyglycinamide, and/or derivatives thereof).

Some of these compounds have been extracted from the HPLC column after glycinamide was incubated in serum, as described above, and identified by mass spectrometry and nuclear magnetic resonance (NMR) spectrometry. These compounds and derivatives or related compounds can be synthesized from available starting materials, as described below.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility or stability in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated by reference herein in its entirety, including any drawings.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, the term “alkyl” refers to an aliphatic hydrocarbon group. The alkyl moiety may be a “saturated alkyl” group, which means that it does not contain any alkene or alkyne moieties. The alkyl moiety may also be an “Unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁₋₆ alkyl” or similar designations. By way of example only, “C₁₋₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl (straight chain or branched), and hexyl (straight chain or branched).

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The substituent “R” appearing by itself and without a number designation refers to a substituent selected from the group consisting of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethanesulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group-with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where all of the hydrogen atoms are replaced by halogen atoms.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. The term “aryl” relates to aromatic, preferably benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. The aryl group may be substituted at the para and/or meta positions. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl 3-cyanophenyl, 4-cyanophenyl, dimethylphenyl, naphthyl, hydroxynaphthyl, hydroxymethylphenyl, trifluoromethylphenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

In the present context, the term “heteroaryl” is intended to mean a heterocyclic aromatic group where one or more carbon atoms in an aromatic ring have been replaced with one or more heteroatoms selected from the group comprising nitrogen, sulfur, phosphorous, and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond.

The term “heteroaryl” is understood to relate to aromatic, C₃₋₈ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, preferably connected via one of the ring-forming carbon atoms. Heteroaryl groups may carry one or more substituents, selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, heteroaryl groups may be five- and six-membered aromatic heterocyclic systems carrying 0, 1, or 2 substituents, which may be the same as or different from one another, selected from the list above. Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine and pyrazine, which are all preferred, as well as furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, amino-C₁₋₆-alkyl.

In the present context, the term “alkyl” and “C₁₋₆-alkyl” are intended to mean a linear or branched saturated hydrocarbon chain wherein the longest chain has from one to six carbon atoms, such as methyl, ethyl, n-propyl, isopropyl, n-butyl, isobutyl, sec-butyl, tert-butyl, pentyl, isopentyl, neopentyl, and hexyl. An alkyl chain may be optionally substituted.

The term “heterocyclyl” is intended to mean three-, four-, five-, six-, seven-, and eight-membered rings wherein carbon atoms together with from 1 to 3 heteroatoms constitute said ring. A heterocyclyl may optionally contain one or more unsaturated bonds situated in such a way, however, that an aromatic π-electron system does not arise. The heteroatoms are independently selected from oxygen, sulfur, and nitrogen.

A heterocyclyl may further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings may optionally also be fused to aryl rings, such that the definition includes bicyclic structures. Preferred such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine , maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Binding to the heterocycle may be at the position of a heteroatom or via a carbon atom of the heterocycle, or, for benzo-fused derivatives, via a carbon of the benzenoid ring.

The term “(heterocyclyl)C₁₋₆-alkyl” is understood as heterocyclyl groups connected, as substituents, via an alkyl, each as defined herein. The heterocyclyl groups of (heterocyclyl)C₁₋₆-alkyl groups may be substituted or unsubstituted. The term “(heterocyclyl)C₁₋₆-alkyl” is intended to mean an alkyl chain substituted at least once with a heterocyclyl group, typically at the terminal position of the alkyl chain.

In the present context, the term “C₂₋₈-alkenyl” is intended to mean a linear or branched hydrocarbon group having from two to eight carbon atoms and containing one or more double bonds. Some examples of C₂₋₈-alkenyl groups include allyl, homo-allyl, vinyl, crotyl, butenyl, pentenyl, hexenyl, heptenyl and octenyl. Some examples of C₂₋₈ alkenyl groups with more than one double bond include butadienyl, pentadienyl, hexadienyl, heptadienyl, heptatrienyl and octatrienyl groups as well as branched forms of these. The position of the unsaturation (the double bond) may be at any position along the carbon chain.

In the present context the term “C₂₋₈-alkynyl” is intended to mean a linear or branched hydrocarbon group containing from two to eight carbon atoms and containing one or more triple bonds. Some examples of C₂₋₈-alkynyl groups include ethynyl, propynyl, butynyl, pentynyl, hexynyl, heptynyl and octynyl groups as well as branched forms of these. The position of unsaturation (the triple bond) may be at any position along the carbon chain. More than one bond may be unsaturated such that the “C₂₋₈-alkynyl” is a di-yne or enedi-yne as is known to the person skilled in the art.

In the present context, the term “C₃₋₈-cycloalkyl” is intended to cover three-, four-, five-, six-, seven-, and eight-membered rings comprising carbon atoms only. A C₃₋₈-cycloalkyl may optionally contain one or more unsaturated bonds situated in such a way, however, that an aromatic π-electron system does not arise.

Some examples of preferred “C₃₋₈-cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

The terms “(aryl)C₁₋₆-alkyl” is intended to mean an aryl group connected, as a substituent, via a C₁₋₆-alkyl, each as defined herein. The aryl groups of (aryl)C₁₋₆-alkyl may be substituted or unsubstituted. Examples include benzyl, substituted benzyl, 2-phenylethyl, 3-phenylpropyl, and naphthylalkyl.

The terms “(cycloalkyl)C₁₋₆-alkyl” is intended to mean a cycloalkyl groups connected, as substituents, via an alkyl, each as defined herein.

When used herein, the term “O—C₁₋₆-alkyl” is intended to mean C₁₋₆-alkyloxy, or alkoxy, such as methoxy, ethoxy, n-propoxy, isopropoxy, n-butoxy, isobutoxy, sec-butoxy, tert-butoxy, pentyloxy, isopentyloxy, neopentyloxy and hexyloxy

The term “halogen” includes fluorine, chlorine, bromine and iodine.

In the present context, i.e. in connection with the terms “C₁₋₆-alkyl”, “aryl”, “heteroaryl”, “heterocyclyl”, “C₃₋₆-cycloalkyl”, “heterocyclyl(C₁₋₆-alkyl)”, “(cycloalkyl)alkyl”, “O—C₁₋₆-alkyl”, “C₂₋₈-alkenyl”, and “C₂₋₈-alkynyl”, the term “optionally substituted” is intended to mean that the group in question may be substituted one or several times, such as 1 to 5 times, or 1 to 3 times, or 1 to 2 times, with one or more groups selected from C₁₋₆-alkyl, C₁₋₆-alkoxy, oxo (which may be represented in the tautomeric enol form), carboxyl, amino, hydroxy (which when present in an enol system may be represented in the tautomeric keto form), nitro, alkylsulfonyl, alkylsulfenyl, alkylsulfinyl, C₁₋₆-alkoxycarbonyl, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, C₁₋₆-alkylhydroxyimino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, heterocyclyl, heteroaryl, and halo. In general, the above substituents may be susceptible to further optional substitution.

Unless otherwise indicated, when a substituent is deemed to be “optionally substituted,” it is meant that the substitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In certain embodiments, in the compound of formula A, E is oxygen. In some embodiments, T is also oxygen.

In some embodiments, the term “heterocyclyl” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine , maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula A include those in which R₁ is selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₁ is hydrogen.

In some embodiments, R₂ is selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₂ is hydrogen.

In some embodiments, R₃-R₆ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₃-R₆ are hydrogen.

In further embodiments, R₇ and R₈ are each independently selected from hydrogen and C₁₋₆ alkyl. In some of these embodiments, R₇ and R₈ are hydrogen.

Preferred pharmaceuticals or medicaments consist of, consist essentially of, or comprise a compound of formula C:

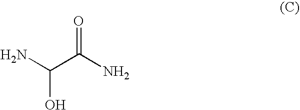

or a pharmaceutically acceptable salt, amide, ester, or prodrug thereof. This compound was isolated using cation exchange HPLC after incubating unmodified G-NH₂ in cofactor-containing serum, as described herein (See EXAMPLE 6). The compound of formula C was identified as modified G-NH₂ (Metabolite X) after the chromatography isolate described above using its NMR spectra.

The analysis was based on a doubly labeled i.e., ¹³C/¹⁵N, sample. The ¹H NMR spectrum consisted of two broad NH-amide signals located at 7.65 and 7.15 ppm and a CH-proton doublet (J=163 Hz) centered at 5.21 ppm. The intensity ratios of all three signals were close to 1:1:1. In the spectrum taken without presaturation of water solvent signal, it was possible to observe extra NH₃⁺ group signal at ˜7.4 ppm. This indicated that one proton in glycine methylene group was replaced by electronegative substituent causing significant downfield shift in ¹H NMR spectrum, as compared to the original glycine amide.

The ¹³C NMR spectrum showed two signals of equal intensity: a doublet for ¹³C═O (J=62 Hz) at 177.6 ppm and eight lines for the aliphatic carbon signal at 89.0 ppm with three different coupling constants (J=7.1; 62 and 163 Hz). J=163 Hz is the one bond ¹³C—¹H coupling, J=62 Hz is the one bond ¹³C—¹³C coupling, while the third coupling 7.1 Hz was in agreement with a one bond ¹⁵N—¹³C coupling. All possible two bond couplings were close to zero as expected from theoretical considerations. Both ¹H—¹³C and ¹³C—¹³C couplings were relatively large, in agreement with the introduction of a strongly electronegative substituent at the glycine aliphatic carbon. The same conclusion came from analysis of the ¹³C chemical shift of that aliphatic carbon, using the existing additive schemes for chemical shift prediction.

¹⁵N—¹H HSQC spectrum consisted of a strong signal from the ¹⁵N labeled amine located ˜20 ppm and a weak signal from unlabelled amide nitrogen at ˜105 ppm. These are expected typical values for NH₃⁺ and CONH₂ nitrogen resonances. The total measurement time for the doubly labeled sample was ˜10 hours.

Thus, the best agreement between the ¹H and ¹³C spectra was obtained for the structure of the compound of formula C. Accordingly, preferred embodiments include pharmaceuticals and medicaments that consist of, consist essentially of (e.g., an enriched or isolated preparation containing the compound of formula C in either enantiomer (D or L) and/or isomer (R or S)), or comprise the compound of formula C and derivatives thereof, in particular, derivatives wherein the hydroxyl group is replaced by a methoxy, ethoxy or alkoxy.

Additional preferred embodiments include pharmaceutical and medicaments that consist of, consist essentially of, or comprise α-peroxyglycinamide dimer (NH₂-gly-O—O-gly-NH₂), having the structure set forth in formula E or diglycinamide ether (NH₂-gly-O-gly-NH₂) having the structure set forth in formula F:

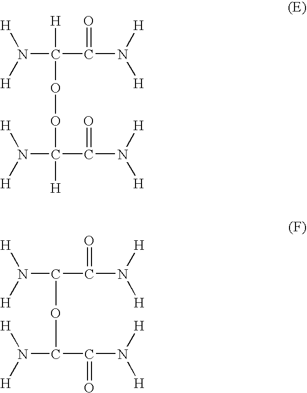

Preferred compositions also include pharmaceuticals and medicaments that consist of, consist essentially of, or comprise alpha-methoxyglycinamide (alpha-MeO-gly-NH₂) having the structure set forth in formula (G):

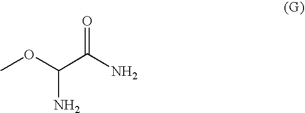

Various approaches to synthesize modified glycinamides are known in the art. (See e.g., JP 5097789A2 to Hayakawa et al., entitled “Alpha-hydroxyglycinamide Derivative and its Preparation,” filed Oct. 3, 1991, herein expressly incorporated by reference in its entirety). By one approach, an α-hydroxyglycinamide derivative represented by the following formula (B) is prepared:

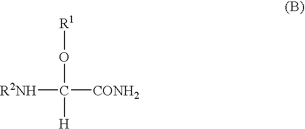

(wherein R¹ is a hydrogen atom, a lower alkyl group, a lower alkenyl group, a lower alkynyl group, a benzyl group, or a silyl group substituted with an alkyl group or an alkyl group and an aromatic group; R² is a hydrogen atom or an amino protecting group) and a salt thereof.

By another approach, an α-hydroxyglycinamide derivative or salt thereof represented by the following formula (H):

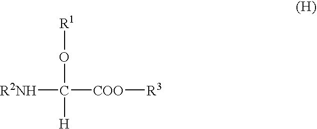

(wherein R¹ and R² are defined in formula (B); R³ is a hydrogen atom or a carboxyl protecting group) is treated with ammonia in a solvent, the amino protecting group is removed if desired, and the compound obtained is further converted into a salt thereof if desired.

In accordance with some of the preferred embodiments described herein, the lower alkyl group represented by reference symbol R₁ is an alkyl group containing no more than 6, preferably no more than 4 carbon atoms. Examples of such groups include methyl group, ethyl group, n-propyl group, isopropyl group, n-butyl group, isobutyl group, tert-butyl group, pentyl group that may be branched, and hexyl group that may be branched.

The lower alkenyl group represented by reference symbol R₁ is an alkenyl group containing no more than 6, preferably no more than 4 carbon atoms. Examples of such groups include ethenyl group, allyl group, and butenyl group having a double bond in any position. The lower alkynyl group represented by reference symbol R₁ is an alkynyl group containing no more than 6, preferably no more than 4 carbon atoms. Examples of such groups include ethynyl group and the like.

The silyl group substituted with a lower alkyl group, which is represented by reference symbol R₁, is a silyl group substituted with 1 to 3 lower alkyl groups. The lower alkyl substituents used in this case are any of the lower alkyl groups described hereinabove with reference to R₁ or combinations thereof. The silyl group substituted with a lower alkyl group is preferably a tert-butyldimethylsilyl group. The silyl group substituted with an alkyl and an aromatic group is a silyl group substituted with the above-described alkyl group and phenyl group, for example, tert-butyldiphenylsilyl group.

Protecting groups that have been used in the field of amino acid or peptide chemistry can be used as the amino protecting group represented by R₂. Examples of such groups include oxycarbonyl-type protecting groups, for example, benzyloxycarbonyl (Cbz-), p-methoxybenzyloxycarbonyl [Z(OMe)-], tert-butoxycarbonyl (Boc-), or 2-biphenylisopropoxycarbonyl (Bpoc-), and the like; acyl protecting groups, for example, HCO—, phthalate group (Pht-), or o-nitrophenylthio group (Nps-), and the like; and alkyl protecting groups, for example, triphenylmethyl group (Trt-), and the like.

Salts of the α-hydroxyglycinamide derivative in accordance with some of the embodiments described herein are acid-added salts, for example, inorganic salts such as hydrohalides, e.g., hydrofluorides, hydrochlorides, hydrobromides, nitrates, sulfates, or phosphates, or organic acid salts such as fumarates, acetates, and the like.

The compounds represented by formula (C) can be prepared by treating an α-hydroxyglycine derivative represented by the following formula (H):

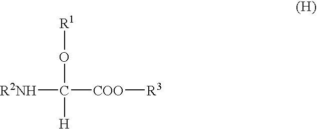

(wherein R¹ and R² are defined in formula (B); R³ is a hydrogen atom or a carboxyl protecting group) with ammonia in a solvent and optionally removing the amino protecting group.

The carbonyl protecting group R³ is an ordinary carboxy protecting group that can be substituted with amino group by treatment with ammonia. Examples of such groups include lower alkyloxy groups, for example, methoxy group (-OMe), ethoxy group (-OEt), benzyloxy group (-OBzl), or tert-butoxy group (-OtBu), or aryloxy group, such as p-nitrophenoxy group (-ONp), and the like.

Ordinary organic solvents such as lower alcohols, for example methanol, ethanol, propanol, ethers such as methyl ethyl ether, diethyl ether, isopropyl ether, and the like can be used as the solvents for the reaction. The reaction can be conducted by dissolving the compound represented by formula (H) in the above-mentioned solvent and blowing ammonia under reduced, normal, or increased pressure at a temperature, for example, from −78° C. to 40° C., preferably from 0° C. to 25° C., e.g. at room temperature.

This reaction makes it possible to obtain the compound (B), in which R² is an amino protecting group. In order to remove the amino protecting group R² from this compound and to obtain the compound (B), in which R² is hydrogen, usual deprotecting treatment may be conducted according to the type of the amino protecting group R². For example, when the protecting group R² is benzyloxycarbonyl, P-methoxybenzyloxycarbonyl, and the like, deprotecting can be carried out by conducting treatment with hydrogen gas in the presence of a hydrogenation catalyst, for example, palladium/carbon or the like. Furthermore, when the protecting group R² is tert-butoxycarbonyl, deprotecting can be conducted with hydrochloric acid-dioxane. A salt of the compound (B) can be produced, for example, by conducting the above-described deprotecting treatment in the presence of an acid such as hydrochloric acid.

A compound according to formula (H), in which R¹ is not a hydrogen atom, can be produced, for example, by the following two methods. With the first method, it can be produced by introducing R¹ other than hydrogen into the compound among the compounds represented by formula (H), in which R¹ is hydrogen. The introduction of the group R¹ other than hydrogen can be conducted with the respective functional derivative of the group, for example, a halogen derivative. For example, for introducing a lower alkyl substituted silyl group, a halide of silyl group can be used, for example, tert-butyldimethylsilyl chloride can be used for introducing a tert-butoxydimethylsilyl group. This reaction can be conducted at a temperature of from 0° C. to 30° C. in a solvent such as dimethylformamide.

Furthermore, in order to introduce a lower alkenyl or lower alkynyl group, a halogen derivative of alkene or alkyl respectively can be used. For example, an allyl group can be introduced by using an allyl halide such as allyl iodide in the presence of a catalyst such as silver oxide. This reaction can be conducted at a temperature from −10 to 50° C., preferably from 0° C. to 25° C., in a solvent such as dimethylformamide.

With the other method for producing the compound of formula (H) in which R¹ is not hydrogen, the compound represented by formula (H) in which both R¹ and R² are hydrogen atoms is treated with thionyl chloride by using a lower alcohol, for example methanol or ethanol as a solvent. In this case, a compound represented by formula (H) in which R¹ and R² are the same lower alkyl group corresponding to the lower alcohol solvent can be obtained. The reaction can be conducted at a temperature from −10° C. to 40° C., preferably from 0° C. to 25° C.

The compound represented by formula (H) in which R¹ is hydrogen can be produced, for example, by the following two methods. With the first method, it can be obtained by reacting glyceraldehydes CHO—COOH with an amine R²NH₂ protected with amino protecting group R². This reaction can be conducted at a temperature of 20° C. to 75° C. in a solvent such as acetone, ether, and the like, for example, by a method described in U.S. Pat. No. 3,668,121 issued to Philip X. Masciantonio et al., and by Stanlen D. Young et al., J. Am. Chem. Soc. 111, 1933 (1989), both of which are expressly incorporated by reference in their entireties. In this case, a compound represented by formula (H) in which both the R¹ and the R³ are hydrogen atoms can be obtained.

With the other method for the preparation of the compound represented by formula (H) in which R¹ is hydrogen, a compound represented by the following formula (I):

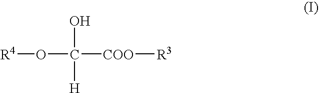

(wherein R³ is defined as described with reference to formula (H), and R⁴ is a lower alkyl group) is reacted with an amine R²NH₂ protected with amino protecting group R². This reaction can be conducted in a solvent such as tetrahydrofuran at a temperature of 20° C. to 80° C., for example, at the reflux temperature of the solvent used. The lower alkyl group R⁴ is defined as the lower alkyl group R¹. The following examples describe some of these synthetic approaches in greater detail.

### Example 12

### 12-1

α-Hydroxy-N-tert-butoxycarbonylglycine methyl ester (4.11 g, 20 mmol) and imidazole are dissolved in DMF at room temperature and cooled to a temperature of 0° C. Then chlorinated tert-butyldimethylsilyl is added to the solution at this temperature and the components are stirred for 10 min. The solution is returned to room temperature and stirring is continued for 1 hour. Then, saturated brine is added and extraction is conducted with ethyl acetate. The organic layer is dried with anhydrous magnesium sulfate and the solvent is distilled off.

The oily substance obtained is then dissolved in ethanol (50 mL) and excess ammonia is blown into the solution at a temperature of 0° C. Next, the excess ammonia is removed under reduced pressure and ethanol is distilled off. The crude product thus obtained is purified by silica gel column chromatography and α-tert-butyldimethylsilyloxy-N-tert-butoxycarbonylglycinamide (6.10 g, quant.) is obtained. An expected profile includes: ¹HNMR d(CDCl₃) 0.16 (s, 3H), 0.21 (s, 3H), 0.92 (s, 9H), 5.46 (d, 1H, J=9 Hz), 5.63 (d, 1H, J=9 Hz), 6.22-6.82 (br, 2H).

### 12-2

The α-hydroxy-N-tert-butoxycarbonylglycine methyl ester that is a starting substance in 12-1 above is prepared in the manner as follows: tert-Butyl carbamate (2.83 g, 23.6 mmol) and glyoxylic acid monohydrate (2.02 g, 21.5 mmol) are dissolved in acetone (50 mL) and refluxed overnight. The solution is then cooled to a temperature of 0° C. and treated with excess diazomethane-ether solution at this temperature. The solvent is then distilled off.

Saturated brine is then added, extraction is conducted with chloroform, the organic layer is dried with anhydrous magnesium sulfate and the solvent is distilled off. The crude product thus obtained is purified by silica gel column chromatography and α-hydroxy-N-tert-butoxycarbonylglycine methyl ester (2.56 g, 58%) is obtained. An expected profile includes: ¹HNMR d(CDCl₃) 1.46 (s, 9H), 1.65 (br s, 1H), 3.84 (s, 3H), 5.27-5.52 (br, 1H), 5.59-5.90 (br, 1H). IR(NaCl) 1755 (s), 1690 (s), 1528 (s) cm⁻¹.

### 12-3

The α-hydroxy-N-tert-butoxycarbonylglycine methyl ester that is a starting substance in 12-1 above can be prepared by a method other than that of 12-2. Accordingly, tert-Butyl carbamate (11.35 g, 95.0 mmol) and 1-hydroxy-1-methoxyacetic acid methyl ester (14.35 g, 119.5 mmol) are dissolved in anhydrous THF (50 mL) and refluxed overnight. The temperature is then returned to room temperature, 1-hydroxy-1-methoxyacetic acid methyl ester (1.15 g, 9.6 mmol) is then added and the components are further refluxed for 8 h. The reaction liquid is allowed to sit until the temperature returns to room temperature and the solvent is then distilled off. The crude product thus obtained is recrystallized from a chloroform-hexane solution and pure α-hydroxy-N-tert-butoxycarbonylglycine methyl ester (16.42 g, 84%) is obtained.

### Example 13

The α-hydroxy-N-tert-butoxycarbonylglycine methyl ester (1.21 g, 5.9 mmol) obtained in 12-2 or 12-3 above is dissolved in DMF (10 mL), and then silver oxide (1.04 g, 4.5 mmol) and benzene iodide (1.99 g, 9.1 mmol) are added at room temperature. The components are stirred overnight at room temperature, the precipitate is filtered, water is added to the mother liquor, and extraction is conducted with ethyl acetate. The extracted solution is dried with anhydrous magnesium sulfate, then the solvent is distilled off and crude purification is conducted with silica gel column chromatography.

The oily substance thus obtained is dissolved in ethanol (50 mL) and excess ammonia is blown into the solution at a temperature of 0° C. The excess ammonia is then removed under reduced pressure and the solvent is distilled off. The crude product thus obtained is purified by silica gel column chromatography and α-benzyloxy-N-tert-butoxycarbonylglycinamide (0.397 g, 22%) is obtained. An expected profile includes: m.p. 115-120° C., ¹HNMR d(CDCl₃) 1.44 (s, 9H), 4.61 (d, 1H, J=11.3 Hz), 4.79 (d, 1H, J=11.3 Hz), 5.4 (d, 1H, J=9.0 Hz), 5.75 (brd, 1H, J=9.0 Hz), 6.00 (br, 1H), 6.52 (br, 1H), 7.35 (s, 5H). IR(NaCl) 1698 (s), 1664 (s), 1502 (s), 732 (m), 695 (m) cm⁻¹. Analytical values for elements (C₁₄H₂₀O₄N₂): Calcd. C, 59.99; H, 7.19; N, 9.99 Obsd. C, 59.94; H, 7.33; N, 10.28 are expected.

### Example 14

The α-hydroxy-N-tert-buthoxycarbonylglycinemethyl ester (2.07 g, 10.1 mmol) prepared according to 12-2 or 12-3 above is dissolved in DMF (20 mL), and silver oxide (1.39 g, 6.0 mmol) and allyl iodide (1.2 mL, 12.9 mmol) are added at room temperature. After overnight stirring at room temperature, the precipitate is filtered out, water is added to the mother liquor, and extraction with ethyl acetate is conducted. The extracted solution is dried with anhydrous magnesium sulfate, then the solvent is distilled off, and an aqueous solution of sodium thiosulfate is added, followed by extraction with ethyl acetate and removal of iodine as a reaction byproduct.

The oily substance thus obtained is dissolved in ethanol, excess ammonia is blown into the solution at a temperature of 0° C., the excess ammonia is thereafter removed under reduced pressure, and the solvent is distilled off. The crude product obtained is purified with silica gel column chromatography to obtain α-allyloxy-N-tert-butoxycarbonylglycinamide (0.625 g, 27%). An expected profile includes: ¹HNMR d(CDCl₃) 1.45 (s, 9H), 4.14 (dd, 2H, J=7.2, 1.8 Hz), 5.11-5.56 (m, 3H), 5.70-6.20 (m, 2H), 6.33-7.01 (m, 2H). IR(CDCl₃) 2975 (w), 1705 (s, br), 1498 (m), 990 (sh, w) cm⁻¹.

### Example 15

### 15-1

α-Hydroxy-N-benzyloxycarbonylglycine (4.44 g, 19.7 mmol) is dissolved in methanol (20 mL). Thionyl chloride (2.9 mL, 40.0 mmol) is dropwise added to the solution at a temperature of 0° C., and stirring is conducted for 30 minutes at this temperature and then for 2 hours at room temperature. The solvent is then distilled off and the crude product obtained is dissolved in methanol (50 mL). The solution is cooled to 0° C., and excess ammonia is blown therein.

Upon completion of the reaction, the excess ammonia is removed under reduced pressure, the solvent is distilled off, and the white crystals obtained are purified with silica gel column chromatography to obtain α-methoxy-N-benzyloxycarbonylglycinamide (3.42 g, 73%). An expected profile includes: m.p. 110-112° C., ¹HNMR d(CDCl₃) 3.44 (s, 3H), 5.16 (s, 2H), 5.31 (d, 1H, J=8.8 Hz), 5.45-5.98 (br, 2H), 6.28-6.68 (br, 1H), 7.36 (s, 5H). IR(NaCl) 1680 (s, br), 1540 (s), 1520 (s), 860 (m), 700 (m) cm⁻¹. Analytical values of elements (C₁₁H₁₄O₄N₂); Calcd. C, 55.46; H, 5.92; N, 11.76 Obsd. C, 55.70, H, 5.94, N, 11.58 are expected.

### 15-2

The α-hydroxy-N-benzyloxycarbonylglycine that is the starting material in 12-4 above is prepared in the manner as follows. Benzyl carbamate (30.24 g, 0.2 mol) and glyoxylic acid monohydrate (20.26 g, 0.22 mol) are dissolved in diethyl ether (200 mL) and the solution is stirred overnight at room temperature. The crystals produced are filtered and then washed with ether to obtain pure α-hydroxy-N-benzyloxycarbonylglycine (33.78 g, 75%). An expected profile includes: m.p. 200-205° C., ¹HNMR d(CD₃OD) 5.12 (s, 2H), 5.40 (s, 1H), 7.34 (s, 5H).

### Example 16

The α-hydroxy-N-benzyloxycarbonylglycine (2.26 g, 10.0 mmol) produced according to 15-2 above is dissolved in ethanol (20 mL). Thionyl chloride (2 mL, 27.4 mmol) is dropwise added to the solution at a temperature of −10° C., and stirring is conducted overnight at room temperature. The solvent is then distilled off and the crude product thus obtained is purified with silica gel column chromatography to obtain α-ethoxy-N-benzyloxycarbonylglycine ethyl ester (2.81 g, quant.). An expected profile includes; m.p. 66-68° C., ¹HNMR d(CDCL₃) 1.22 (t, 3H, J=7.2 Hz), 1.30 (t, 3H, J=7.2 Hz), 3.70 (q, 2H, J=7.2 Hz), 4.24 (q, 2H, J=7.2 Hz), 5.15 (s, 2H), 5.33 (d, 1H, J=9.7 Hz), 5.93 (brd, 1H, J=9.7 Hz), 7.35 (s, 5H). IR(NaCl) 1740 (s), 1700 (s), 1540 (s), 760 (m), 700 (m) cm⁻¹. Analytical values of elements (C₁₄H₁₉O₅N); Calcd. C, 59.78, H, 6.81; N, 4.98, Obsd. C, 60.03; H, 6.88; N, 4.89 are expected.

### Example 17

The α-hydroxy-N-benzyloxycarbonylglycine (2.26 g, 10.0 mmol) produced according to 15-2 above is dissolved in isopropyl alcohol (20 mL). Thionyl chloride (2 mL, 27.4 mmol) is dropwise added to the solution at a temperature of −10° C., and stirring is conducted overnight at room temperature. The solvent is then distilled off and the crude product thus obtained is purified with silica gel column chromatography to obtain α-isopropoxy-N-benzyloxycarbonylglycine isopropyl ester (3.10 g, quant.). An expected profile includes: ¹HNMR d(CDCL₃) 1.16-1.37 (m, 12H), 3.87-4.22 (m, 1H), 4.57-5.20 (m, 1H), 5.14 (s, 2H), 5.33 (d, 1H, J=9.7 Hz), 5.93 (brd, 1H, J=9.7 Hz), 7.35 (s, 5H). IR(Neat) 1728 (s, br), 1508 (m), 740 (m) cm⁻¹.

### Example 18

The α-ethoxy-N-benzyloxycarbonylglycine ethyl ester (2.29 g, 8.1 mmol) produced according to EXAMPLE 16 is dissolved in ethanol (80 mL) and cooled to 0° C. Excess ammonia is then blown into the solution at this temperature. Upon completion of the reaction, the excess ammonia is removed under reduced pressure, the solvent is distilled off, and the white crystals thus obtained are washed with a hexane-ethyl acetate mixed solution to obtain pure α-ethoxy-N-benzyloxycarbonylglycinamide (1.51 g, 77%). An expected profile includes: m.p 119-121° C., ¹HNMR d(CDCL₃) 1.23 (t, 3H, J=7.1 Hz), 3.50-3.90 (m, 2H), 5.14 (s, 2H), 5.37 (d, 1H, J=9.0 Hz), 5.65-5.96 (br, 2H), 6.41-6.71 (br, 1H), 7.35 (s, 5H). IR(NaCl) 1680 (s), 1664 (s), 1542 (m), 1524 (m), 760 (w), 740 (w), 700 (m) cm⁻¹. Analytical values of elements (C₁₂H₁₆O₄N₂); Calcd. C, 57.13; H, 6.39; N, 11.10, Obsd. C, 57.09; H, 6.34, N, 11.37 are expected.

### Example 19

The α-isopropoxy-N-benzyloxycarbonylglycine isopropyl ester (2.48 g, 8.0 mmol) produced according to EXAMPLE 16 is dissolved in ethanol (40 mL) and cooled to 0° C. Then, excess ammonia is blown into the solution for 5 hours at this temperature and stirring is further conducted for 2 days in the ammonia saturated state. Upon completion of the reaction, the excess ammonia is removed under reduced pressure, the solvent is distilled off, and the white crystals thus obtained are washed with a hexane—ethyl acetate mixed solution to obtain pure α-isopropoxy-N-benzyloxycarbonylglycinamide (1.64 g, 77%). An expected profile includes: m.p 111-113° C., ¹HNMR d(CDCL₃) 1.18 (d, 3H, J=4.4 Hz), 1.25 (d, 3H, J=4.4 Hz), 3.81-4.20 (m, 1H), 5.15 (s, 2H), 5.44 (d, 1H, J=9.0 Hz), 5.53-5.86 (br, 2H), 6.37-6.73 (br, 1H), 7.35 (s, 5H). IR(NaCl) 1668 (s), 1660 (s), 1538 (m), 1530 (m), 760 (w), 740 (w), 700 (m) cm⁻¹. Analytical values of elements (C₁₃H₁₈O₄N₂); Calcd. C, 58.63; H, 6.81; N, 10.52. Obsd. C, 58.60; H, 6.82; N, 10.54 are expected.

### Example 20

The α-tert-butyldimethylsilyloxy-N-tert-buthoxycarbonylglycinamide (5.08 g, 16.7 mmol) produced according to (12-1) of EXAMPLE 12 is dissolved in dioxane (10 mL) and cooled to 0° C. Then, a 4N hydrochloric acid-dioxane solution (17 mL) is added and stirring is conducted for 1 hour at this temperature.

In order to complete the reaction, a 4N hydrochloric acid-dioxane solution is further added, the temperature is raised to room temperature and stirring is conducted for 1 hour. Diethyl ether is then added to the solution, as large an amount of the product as possible is precipitated, filtered, and washed with ether. The precipitate is then dried under reduced pressure to obtain pure o-hydroxyglycinamide hydrochloride (1.86 g, 88%). An expected profile includes: ¹HNMR d(DMSO-d₆) 4.99 (br sd, 1H), 7.62-8.03 (br, 2H), 8.32-8.85 (br, 3H). IR (KBr) 1686 (s), 1581 (m), 1546 (m), 1477 (s), 843 (m) cm⁻¹

### Example 21

The α-methoxy-N-benzyloxycarbonylglycinamide (0.24 g, 1.0 mmol) prepared according to EXAMPLE 15 (15-1) is dissolved in methanol, 12N hydrochloric acid (0.1 mL) and palladium-carbon (50 mg) are added to the solution at room temperature, and stirring is conducted for 30 minutes under hydrogen atmosphere. The palladium-carbon is then filtered out and the solvent of the mother liquor is distilled off to obtain α-methoxyglycinamide hydrochloride (0.14 g, quant). An expected profile includes: ¹HNMR d(CD₃OD) 3.35 (s, 3H), 5.01 (s, 1H), ¹³CNMR d(CD₃OD) 42.1, 84.3 (d, J=159.8 Hz), 170.3. The next Example describes an approach that was used to synthesize α-hydroxy-glycinamide hydrochloride for formulation into a pharmaceutical or medicament.

### Example 22

### Preparation of α-hydroxy-glycinamide Hydrochloride

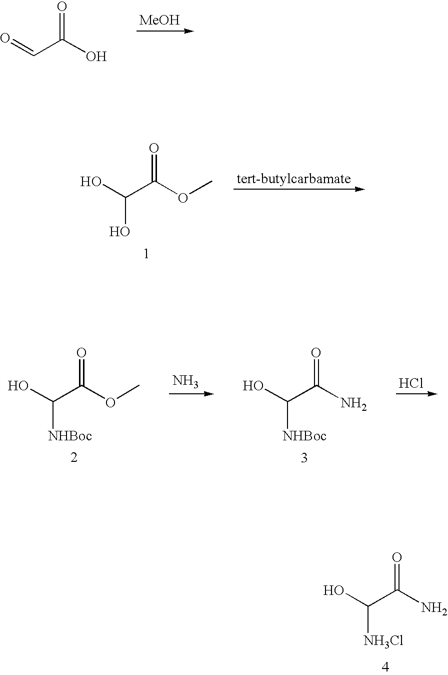

### 22.1 Methyl Glyoxylate Hemiacetal

A solution of glyoxylic acid monohydrate (7.0 g, 76 mmol) in methanol (35 mL) was refluxed overnight. The solution was then neutralized with saturated NaHCO₃ and evaporated. The residue was dissolved in CH₂Cl₂ and dried over Na₂SO₄. Evaporation afforded 3.23 g (40.0%) of crude oil that was used in the following reaction without further purification.

### 22.2 Methyl N-tert-butoxycarbonyl-α-hydroxyglycinate

A solution of methyl glyoxylate hemiacetal (2.0 g, 18.9 mmol) and tert-butyl carbamate (2.0 g, 17.18 mmol) in toluene (45 mL) was refluxed overnight. Evaporation afforded oil. This crude oil was purified by silica gel chromatography EtOAc/heptane 1/9 to 2/8 as eluent. The pure fractions gave 0.6 g oily product that was then crystallized with diethyl ether/heptane. The yield 0.39 g (10.1%). The NMR spectra observed were:

¹H NMR (300 MHz, CDCl₃) δ 5.74 (br s, 1H), 5.44 (br s, 1H), 3.84 (s, 3H), 1.46 (s, 9H).

¹³C NMR (300 MHz, DMSO-d₆) δ 170.3, 154.7, 78.6, 72.8, 51.9, 28.1.

### 22.3 N-tert-butoxycarbonyl-α-hydroxyglycinamide

Methyl N-tert-butoxycarbonyl-α-hydroxyglycinate (0.34 g, 1.66 mmol) was solved in 7N NH₃ in methanol (4 mL). The solution was stirred at room temperature overnight, evaporated and then co-evaporated twice with acetonitrile. The product was purified by silica gel chromatography EtOAc/heptane 3/7 to 5/5 as eluent. The yield 0.1 g (31.7%). The NMR spectra observed were:

¹H NMR (300 MHz, DMSO-d₆) δ 7.28 (br d, 2H), 6.20 (d, 1H), 5.09 (t, 1H), 1.39 (s, 9H).

¹³C NMR (300 MHz, DMSO-d₆) δ 171.7, 155.0, 78.3, 73.4, 28.2.

### 22.4 a-Hydroxy-glycinamide Hydrochloride

N-tert-butoxycarbonyl-α-hydroxyglycinamide (40 mg, 0.2 mmol) was solved in dioxane (1.5 mL). 4N HCl in dioxane (0.5 mL) was added to the solution at 0° C. The cooling bath was removed and the solution was stirred for 40 min. at room temperature. Diethyl ether was added and the solution was stirred. Ether was decanted and the residue was evaporated. The yield was approximately ˜40 mg. The NMR spectra observed were:

¹H NMR (500 MHz, DMSO-d₆) δ 8.5-7.1 (m, 5H), 4.85 (s, 1H).

¹³C NMR (500 MHz, DMSO-d₆) δ 173.1, 87.4.

The following Example describes an approach that was used to prepare α-methoxy-glycinamide.

### Example 23

### Preparation of α-Methoxy-glycinamide

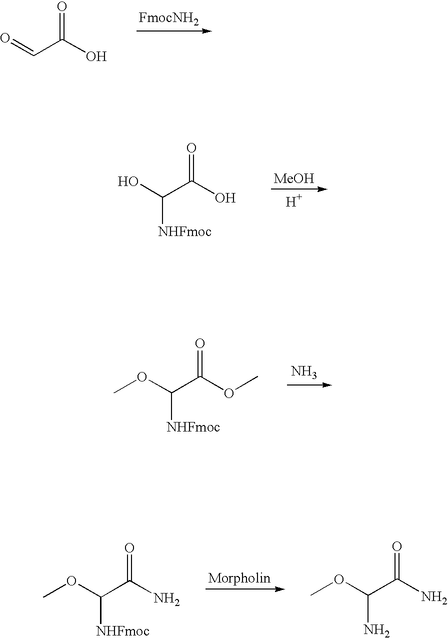

### 23-1 Methyl N-(9H-Fluoren-9-ylmethoxycarbonyl)-a-methoxyglycinate

Glyoxylic acid monohydrate (276 mg, 3 mmol) and 9H-fluoren-9-ylmethyl carbamate (320 mg, 1.33 mmol) were solved in dry diethylether (10 mL). The mixture was stirred at room temperature overnight. The solvent was evaporated and the residue was solved in methanol (20 mL) and 1 drop of sulfuric acid was added. The reaction mixture was stirred 3 days at room temperature. Sat. NaHCO₃ (100 mL) was added to the mixture and it was extracted with ethyl acetate, dried over Na₂SO₄ and evaporated. The residue was purified on silica gel column to give 250 mg (55%) of the titled compound. The NMR spectra observed were:

¹H NMR (300 MHz, CDCl₃) δ 7.76 (d, 2H), 7.59 (d, 2H), 7.40 (t, 2H), 7.31 (t, 2H), 5.90 (br d, 1H), 5.35 (d, 1H), 4.46 (m, 2H), 4.24 (t, 1H), 3.82 (s, 3H), 3.43 (s, 3H).

¹³C NMR (300 MHz, CDCl₃) δ 143.6, 143.5, 141.2, 127.7, 127.1, 124.9, 120.0, 80.5, 67.2, 56.2, 52.9.

### 23-2 a-Methoxyglycinamide

Methyl N-(9H-Fluoren-9-ylmethoxycarbonyl)-a-methoxyglycinate (240 mg, 0.7 mmol) was treated with 3N NH₃ in methanol (20 mL) at room temperature overnight. Methanol was removed by evaporation. The solid was solved in THF (30 mL) and morpholine (305 mg, 3.5 mmol) was added. The mixture was stirred at room temperature for 5 h. The solvent was evaporated and the product was purified on silica gel column to give 5 mg (6%) of the titled compound. The NMR spectrum observed was:

1H NMR (300 MHz, CDCl₃) δ 4.40 (br s, 1H), 3.35 (s, 3H).

The modified glycinamide compounds described herein are suitable for use as a biotechnological tool to study the interaction of the compound with HIV and also as a pharmaceutical or medicament for the treatment of subjects already infected with HIV, or as a preventive preparation to avoid HIV infection. The cofactor(s) obtainable by the methods described herein (either alone or in conjunction or combination with G-NH₂ or a G-NH₂ containing peptide, such as GPG-NH₂) are also suitable for use as biotechnological tools and as medicaments for the treatment and prevention of HIV replication. By one approach, for example, a prodrug therapy is contemplated, wherein G-NH₂ or a G-NH₂ containing peptide, such as GPG-NH₂, is provided to a subject in need and the cofactor is provided by co-administration. Alternatively, the G-NH₂ or a G-NH₂ containing peptide, such as GPG-NH₂ and the cofactor can be combined in a pharmaceutical (e.g., a pharmaceutical composition comprising G-NH₂ or a G-NH₂ containing peptide, such as GPG-NH₂, and the cofactor). In this vein, cofactor and/or G-NH₂ and/or GPG-NH₂ and/or other glycinamide containing peptides can be administered as prodrugs when, for example, time release or long term treatments are desired.

Although anyone could be treated with these anti-HIV compositions as a prophylactic, the most suitable subjects are people at risk for viral infection. Such subjects include, but are not limited to, the elderly, the chronically ill, homosexuals, prostitutes, intravenous drug users, hemophiliacs, children, and those in the medical profession who have contact with patients or biological samples.

Methods of making and using medicaments comprising modified G-NH₂ (e.g., Metabolite X or AlphaHGA) are also embodiments of the present invention. The modified G-NH₂ obtainable by the methods described herein can be processed in accordance with conventional methods of galenic pharmacy to produce medicinal agents for administration to patients, e.g., mammals including humans. The modified G-NH₂ can be incorporated into a pharmaceutical product with and without modification. Further, the manufacture of pharmaceuticals or therapeutic agents that deliver modified G-NH₂ by several routes is included within the scope of the present invention.

The modified G-NH₂ described herein can be employed in admixture with conventional excipients, i.e., pharmaceutically acceptable organic or inorganic carrier substances suitable for parenteral, enteral (e.g., oral) or topical application that do not deleteriously react with the peptide agents. Suitable pharmaceutically acceptable carriers include, but are not limited to, water, salt solutions, alcohols, gum arabic, vegetable oils, benzyl alcohols, polyethylene glycols, gelatine, carbohydrates such as lactose, amylose or starch, magnesium stearate, talc, sialicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the modified G-NH₂.

In some embodiments, medicaments comprising modified G-NH₂ are formulated with or administered in conjunction with other agents that inhibit viral infections, such as HIV infection, so as to achieve a better viral response. At present four different classes of drugs are in clinical use in the antiviral treatment of HIV-1 infection in humans. These are (i) nucleoside analogue reverse transcriptase inhibitors (NRTIs), such as zidovudine, lamivudine, stavudine, didanosine, abacavir, and zalcitabine; (ii) nucleotide analogue reverse transcriptase inhibitors, such as tenofovir; (iii) non-nucleoside reverse transcriptase inhibitors (NNRTIs), such as efavirenz, nevirapine, and delavirdine; and (iv) protease inhibitors, such as indinavir, nelfinavir, ritonavir, saquinavir and amprenavir. By simultaneously using two, three, or four different classes of drugs in conjunction with administration of the modified G-NH₂, HIV is less likely to develop resistance, since it is less probable that multiple mutations that overcome the different classes of drugs and the modified G-NH₂ will appear in the same virus particle.

It is thus preferred that medicaments comprising modified G-NH₂ are formulated with or given in combination with nucleoside analogue reverse transcriptase inhibitors, nucleotide analogue reverse transcriptase inhibitors, non-nucleoside reverse transcriptase inhibitors, and protease inhibitors at doses and by methods known to those of skill in the art. Medicaments comprising the modified G-NH₂ and nucleoside analogue reverse transcriptase inhibitors, nucleotide analogue reverse transcriptase inhibitors, non-nucleoside reverse transcriptase inhibitors, and protease inhibitors can be formulated to contain other ingredients to aid in delivery, retention, or stability of the modified G-NH₂.

The effective dose and method of administration of a particular modified G-NH₂ formulation can vary based on the individual patient and the stage of the disease, as well as other factors known to those of skill in the art. Therapeutic efficacy and toxicity of such compounds can be determined by standard pharmaceutical procedures in cell cultures or experimental animals, e.g., ED₅₀ and LD₅₀ (the dose lethal to 50% of the population). The dose ratio of toxic to therapeutic effects is the therapeutic index, and it can be expressed as the ratio, LD₅₀/ED₅₀. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, age, weight and gender of the patient; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Normal dosage amounts may vary from approximately 1 to 100,000 micrograms, up to a total dose of about 20 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, 10 g, 11 g, 12 g, 13 g, 14 g, 15 g, 16 g, 17 g, 18 g, 19 g, and 20 g. Additionally, the concentrations of the modified G-NH₂ can be quite high in embodiments that administer the agents in a topical form. Molar concentrations of peptide agents can be used with some embodiments. Desirable concentrations for topical administration and/or for coating medical equipment range from 100 μM to 800 mM. Preferable concentrations for these embodiments range from 500 μM to 500 mM. For example, preferred concentrations for use in topical applications and/or for coating medical equipment include 500 μM, 550 μM, 600 μM, 650 μM, 700 μM, 750 μM, 800 μM, 850 μM, 900 μM, 1 mM, 5 mM, 10 mM, 15 mM, 20 mM, 25 mM, 30 mM, 35 mM, 40 mM, 45 mM, 50 mM, 60 mM, 70 mM, 80 mM, 90 mM, 100 mM, 120 mM, 130 mM, 140 mM, 150 mM, 160 mM, 170 mM, 180 mM, 190 mM, 200 mM, 300 mM, 325 mM, 350 mM, 375 mM, 400 mM, 425 mM, 450 mM, 475 mM, and 500 mM. Guidance as to particular dosages and methods of delivery is provided in the literature and below. (See e.g., U.S. Pat. Nos. 4,657,760; 5,206,344; or 5,225,212, herein expressly incorporated by reference in their entireties).

More specifically, the dosage of the modified G-NH₂ is one that provides sufficient modified G-NH₂ to attain a desirable effect including inhibition of proper viral release and/or inhibition of HIV replication. Accordingly, the dose of modified G-NH₂ preferably produces a tissue or blood concentration or both from approximately 0.1 nM to 500 mM. Desirable doses produce a tissue or blood concentration or both of about 0.1 nM to 800 μM. Preferable doses produce a tissue or blood concentration of greater than about 10 nM to about 300 μM. Preferable doses are, for example, the amount of modified G-NH₂ required to achieve a tissue or blood concentration or both of 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 200 nM, 300 nM, 400 nM, 500 nM, 600 nM, 700 nM, 800 nM, 900 nM, 1 nM, 10 nM, 15 nM, 20 nM, 25 nM, 30 nM, 50 μM, 100 μM, 200 μM, and 300 μM. Although doses that produce a tissue concentration of greater than 800% are not preferred, they can be used with some embodiments. A constant infusion of the modified G-NH₂ can also be provided so as to maintain a stable concentration in the tissues as measured by blood levels.

Routes of administration of the modified G-NH₂ include, but are not limited to, topical, transdermal, parenteral, gastrointestinal, transbronchial, and transalveolar. Topical administration is accomplished via a topically applied cream, gel, rinse, etc. containing modified G-NH₂. Transdermal administration is accomplished by application of a cream, rinse, gel, etc. capable of allowing the modified G-NH₂ to penetrate the skin and enter the blood stream. Parenteral routes of administration include, but are not limited to, electrical or direct injection such as direct injection into a central venous line, intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally.

Compositions of modified G-NH₂ containing compounds suitable for topical application include, but are not limited to, physiologically acceptable implants, ointments, creams, rinses, and gels. Any liquid, gel, or solid pharmaceutically acceptable base in which the compounds are at least minimally soluble is suitable for topical use in the present invention. Compositions for topical application are particularly useful during sexual intercourse to prevent transmission of HIV. Suitable compositions for such use include, but are not limited to, vaginal or anal suppositories, creams, jellies, lubricants, oils, and douches.

Compositions of the modified G-NH₂ suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference and are well known in the art. Examples of suitable transdermal devices are described, for instance, in U.S. Pat. No. 4,818,540, issued Apr. 4, 1989 to Chinen, et al., hereby incorporated by reference in its entirety.

Compositions of the modified G-NH₂ suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, or subcutaneous injection of the modified G-NH₂.

Compositions of the modified G-NH₂ suitable for transbronchial and transalveolar administration include, but are not limited to, various types of aerosols for inhalation. For instance, pentamidine is administered intranasally via aerosol to AIDS patients to prevent pneumonia caused by pneumocystis carinii. Devices suitable for transbronchial and transalveolar administration of the modified G-NH₂, including but not limited to atomizers and vaporizers, are also included within the scope of the present invention. Many forms of currently available atomizers and vaporizers can be readily adapted to deliver modified G-NH₂.

Compositions of the modified G-NH₂ suitable for gastrointestinal administration include, but not limited to, pharmaceutically acceptable powders, pills, sachets, or liquids for ingestion and suppositories for rectal administration. Due to the most common routes of HIV infection and the ease of use, gastrointestinal administration, particularly oral, is preferred. Pharmaceuticals for gastrointestinal administration, for example, are formulated in capsule, pill, or tablet form, wherein the active ingredient, modified glycinamide (e.g., α-hydroxyglycinamide, α-peroxyglycinamide dimer, diglycinamide ether, or α-methoxyglycinamide), is in an amount effective to inhibit HIV replication.

The modified G-NH₂ is also suitable for use in situations where prevention of HIV infection is important. For instances, medical personnel are constantly exposed to patients who may be HIV positive and whose secretions and body fluids contain the HIV virus. Further, the modified G-NH₂ can be formulated into antiviral compositions for use during sexual intercourse so as to prevent transmission of HIV. Such compositions are known in the art and also described in the international application published under the PCT publication number WO90/04390 on May 3, 1990 to Modak et al., which is incorporated herein by reference in its entirety.

Embodiments of the invention also include a coating for medical equipment such as gloves, sheets, and work surfaces that protects against viral transmission. Alternatively, the modified G-NH₂ can be impregnated into a polymeric medical device. Particularly preferred are coatings for medical gloves and condoms. Coatings suitable for use in medical devices can be provided by a powder containing the peptides or by polymeric coating into which the peptide agents are suspended. Suitable polymeric materials for coatings or devices are those that are physiologically acceptable and through which a therapeutically effective amount of the modified G-NH₂ can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, issued Sep. 16, 1986 to Fox et al., which is incorporated herein by reference in its entirety. Accordingly, methods of making a medicament that inhibits HIV replication involve providing modified G-NH₂ and formulating said medicament for delivery to a subject, including a human, as described above.

Methods of identification of compounds that inhibit HIV replication are also provided. By one method, for example, a compound for incorporation into an anti-HIV pharmaceutical is identified by incubating G-NH₂ with serum, plasma, or a cell extract for a time sufficient to metabolize modified G-NH₂ and isolating the modified G-NH₂ by cation exchange HPLC. Preferably, human sera, pig sera, bovine sera, cat sera, dog sera, horse sera, monkey sera, or pig plasma is used. By this approach, modified G-NH₂ rapidly elutes from the column, whereas unreacted G-NH₂ is retained on the column for a considerably longer period of time. The isolation of modified G-NH₂ can be further confirmed by conducting HIV infectivity studies in the presence of the isolated compound, as described above. Similarly, synthetic compounds that are related to α-hydroxyglycinamide, α-peroxyglycinamide dimer, diglycinamide ether, methoxyglycinamide, α-ethoxyglycinamide, and derivatives of these compounds can be screened using the HIV infectivity studies presented herein. Depending on the purity of the modified G-NH₂ isolated or the structure of the synthetic modified glycinamide, the ED₅₀ of the compound is between less than 1 μM and less than 30 μM. That is, the ED₅₀ of pure modified G-NH₂ is less than 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM. Thus, in some embodiments, the modified G-NH₂ identified by the methods above is incorporated in a pharmaceutical. Furthermore, the methods above can be supplemented by providing an antiviral compound selected from the group consisting of nucleoside analogue reverse transcriptase inhibitors, nucleotide analogue reverse transcriptase inhibitors, non-nucleoside reverse transcriptase inhibitors, and protease inhibitors into the pharmaceutical. Additionally, the methods above can be supplemented by incorporating a carrier into the pharmaceutical.

Although the modified G-NH₂ can be used as a research tool to analyze the inhibition of HIV, desirably modified G-NH₂ is used to inhibit HIV replication and infection in a subject. By one method, for example, a subject at risk of becoming infected by HIV or who is already infected with HIV is identified and said subject is provided modified G-NH₂. By an additional method, a subject is provided modified G-NH₂ and the effect on HIV replication or infection, is determined (e.g., by analyzing the amount of p24 or reverse transcriptase activity in a biological sample).

It is contemplated that modified glycinamide inhibits replication of HIV by a mechanism that is different than conventional nucleoside analogues and protease inhibitors. (See U.S. Pat. Nos. 6,258,932; 6,455,670; 6,537,967; all of which are hereby expressly incorporated by reference in their entireties). Accordingly, preferred subjects to receive pharmaceuticals containing modified glycinamide are HIV infected individuals that have developed resistance to nucleoside analogues and protease inhibitors.

By one approach, nine HIV infected patients are provided differing amounts of modified glycinamide (e.g., alpha-hydroxyglycinamide, alpha-peroxyglycinamide dimer, diglycinamide ether or alpha-methoxyglycinamide) and the inhibition of HIV replication is analyzed. Group I, which contains three individuals, is provided 1.0 g of modified glycinamide by capsule form three times a day; whereas Group II, which contains three individuals, is provided 1.5 g of modified glycinamide by capsule form three times a day; and Group III, which contains three individuals is provided 2.0 g of modified glycinamide by capsule form throughout the day. The reduction in viral lode is monitored daily by conventional techniques that detect the amount of HIV RNA (e.g., Roche AMPLICOR MONITOR™). A reduction in viral lode will be observed, as indicated by a reduction in the amount of HIV RNA detected.

The methods above can be supplemented with administration of an antiviral treatment selected from the group consisting of nucleoside analogue reverse transcriptase inhibitors, nucleotide analogue reverse transcriptase inhibitors, non-nucleoside reverse transcriptase inhibitors, and protease inhibitors. Further, the modified G-NH₂ used in these methods can be joined to a support or can be administered in a pharmaceutical comprising a pharmaceutically acceptable carrier.

While the present invention has been described in some detail for purposes of clarity and understanding, one skilled in the art will appreciate that various changes in form and detail can be made without departing from the true scope of the invention. All figures and tables, as well as patents, applications, and publications referred to above are hereby expressly incorporated by reference in their entireties.

What is claimed is:

1. A method of inhibiting in-vitro replication of HIV comprising providing an effective amount of alpha hydroxyglycinamide or a pharmaceutically acceptable salt, amide, ester, or prodrug thereof to a cell culture infected with HIV.

2. The method of claim 1, wherein a compound of formula (C) 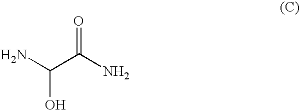
is provided.

3. The method of claim 1, wherein a compound of formula (D) 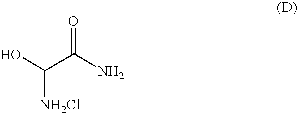
is provided.

4. The method of claim 1, further comprising measuring the presence or absence of HIV or a marker thereof in said cell culture.

5. The method of claim 1, wherein the presence or absence of HIV is measured by a p24 detection assay.

6. The method of claim 1, wherein the presence or absence of HIV is measured by reverse transcriptase activity.

7. The method of claim 1, wherein the presence or absence of HIV is measured by syncytia formation.

8. The method of claim 1, wherein said alpha hydroxyglycinamide or pharmaceutically acceptable salt, amide, ester, or prodrug thereof is provided in a septum sealed vial.

9. The method of claim 1, wherein said alpha hydroxyglycinamide or pharmaceutically acceptable salt, amide, ester, or prodrug thereof is provided in a container comprising a certification that said alpha hydroxyglycinamide or pharmaceutically acceptable salt, amide, ester, or prodrug thereof is a good manufacturing practice (GMP) formulation.

10. The method of claim 1, wherein said alpha hydroxyglycinamide or pharmaceutically acceptable salt, amide, ester, or prodrug thereof is provided in a container comprising indicia of approval from a government agency.

11. A method of inhibiting replication of human immunodeficiency virus (HIV) comprising:
identifying a subject in need of a compound that inhibits replication of HIV; and
providing to said subject an HIV inhibiting amount of a composition comprising as an active ingredient, with or without other active ingredients, a compound of alpha hydroxyglycinamide or a pharmaceutically acceptable salt, amide, ester, or prodrug thereof.

12. The method of claim 11, wherein a compound of formula (C) 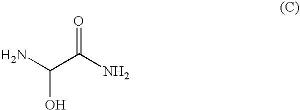
is provided.

13. The method of claim 11, wherein a compound of formula (D) 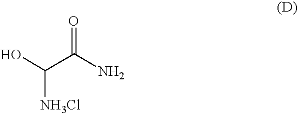
is provided.

14. The method of claim 12, further comprising measuring the inhibition of replication of HIV.

15. The method of claim 12, wherein said composition is formulated for oral administration.
